(19) United States
(12) Patent Application Publication
(10) Pub. No.: US 2010/0311949 A1
(43) Pub. Date: Dec. 9, 2010

(54) ALIGNED COLLAGEN AND METHOD THEREFOR

(75) Inventors: Ozan Akkus, West Lafayette, IN (US); Alyssa Panitch, West Lafayette, IN (US); Xingguo Cheng, San Antonio, TX (US)

Correspondence Address: BARNES & THORNBURG LLP, 11 SOUTH MERIDIAN, INDIANAPOLIS, IN, 46204, US

(73) Assignee: PURDUE RESEARCH FOUNDATION, West Lafayette, IN (US)

(21) Appl. No.: 12/745,362

(22) PCT Filed: Nov. 26, 2008
(86) PCT No.: PCT/US08/84958

§ 371 Date: May 28, 2010

Related U.S. Application Data
(60) Provisional application No. 60/991,536, filed on Nov. 30, 2007.
(60) Provisional application No. 61/048,840, filed on Apr. 29, 2008.

(51) Int. Cl. C07K 14/78

(52) U.S. Cl. 530/356

(57) ABSTRACT

Compositions and methods of preparing a graft construct comprising aligned collagen fibrils, wherein the construct is anisotropic, are described. The construct is prepared by application of an electrochemical field and a pH gradient to solutions containing collagen. In accordance with this method, collagen aligns at its isoelectric point to form anisotropic constructs.

### CROSS REFERENCE TO RELATED APPLICATIONS

This application claims the benefit under 35 U.S.C. §119(e) of U.S. Provisional Application Ser. No. 60/991,536, filed on Nov. 30, 2007, incorporated herein by reference in its entirety. This application also claims the benefit under 35 U.S.C. §119(e) of U.S. Provisional Application Ser. No. 61/048,840, filed on Apr. 29, 2008, incorporated herein by reference in its entirety.

### GOVERNMENT RIGHTS

Research relating to this invention was supported in part by the U.S. Government under Grant No. NSF CAREER-0449188 awarded from the National Science Foundation. The U.S. Government has certain rights in this invention.

### FIELD OF THE INVENTION

The invention relates to compositions and methods of preparing an engineered graft construct comprising aligned collagen fibrils, wherein the construct is anisotropic.

### BACKGROUND AND SUMMARY OF THE INVENTION

Collagen is the major structural protein in connective tissues such as skin, bone, ligaments, and tendons. The specific hierarchical organization of collagen molecules determines the unique properties of each specific tissue. For example, in tendon, the parallel alignment of collagen molecules, fibrils, fibers, fascicles and tendon units gives tendon its unique mechanical properties. Controlled molecular assembly is the process nature uses for the assembly of extracellular matrices with anisotropically oriented collagen fibrils. Controlled assembly of collagen molecules in vitro remains a major challenge for engineering the next generation of tissues.

Collagen has been a key ingredient in, for example, engineered tissue scaffolds, wound healing biomaterials, drug delivery agents, and gene delivery agents. Collagen assembly in vitro is influenced by temperature, pH, ionic strength, solvent forces, and hydrophobic forces. The resulting collagen “gel” is usually a random network of loosely-packed fibers.

As a natural protein, collagen is mainly responsible for the mechanical and structural integrity of load-bearing extracellular matrices of vertebrates and some invertebrates. Rod-like collagen molecules self-assemble in a crystal-like fashion with different packing patterns and the resulting structure is covalently crosslinked to form mechanically strong tissues in vivo. In tendon and ligaments, Type I collagen molecules are anisotropically oriented in a preferred direction resulting in a tissue that exhibits load-bearing properties in a dominant direction.

There have been attempts to make synthetic, engineered collagen structures with some degree of orientational anisotropy. The alignment of collagen molecules by flow, mechanical extrusion, microfluidic channels, or anisotropic chemical nanopatterns has limitations in attaining high packing density, elastic deformability, and in making constructs of relevant sizes. As a consequence, there is currently no practical means to form highly oriented, densely-packed macroscale collagenous constructs on the timescale of minutes to hours.

There are several unique advantages of the electrochemical process described herein: (1) environmentally friendly (no toxic solvents used compared to electrospinning to produce collagen fibers); (2) low cost (low electric voltage and current); (3) practicality of the experimental set up (pair of electrodes in a humid environment), which can be upgradeable to batch processing by employing electrode arrays; (4) ability to produce long rope-like constructs which may be useful in regeneration of longer tendons such as the flexors or extensors of the finger.

The aligned collagen constructs formed by the electrochemical methods described herein are anisotropic and exhibit mechanical properties similar to those of native tendons. Furthermore, the mechanical properties of the resultant collagen fiber bundles, after cross-linking, exhibit an ultimate tensile strength and tensile modulus similar to native tendon. In one embodiment, the mechanical properties of the constructs described herein can be further modified by incorporation of non-collageneous proteins (e.g., fibronectin, fibrinogen, keratin, and silk proteins) and proteoglycans (e.g., decorin) and by increasing the crosslinking density of the constructs. In one embodiment, the constructs described herein may be used to replace load-bearing connective tissues.

In one embodiment, an engineered graft construct is described that comprises aligned collagen fibrils, wherein the construct is anisotropic and the fibril area fraction of the construct is about 80% to about 100%. In accordance with this embodiment, 1) the collagen is crosslinked, 2) the collagen is uncrosslinked, 3) the fibril area fraction of the composition is about 83% to about 100%, 4) the fibril area fraction of the composition is about 86% to about 100%, 5) the fibril area fraction of the composition is about 90% to about 100%, 6) the density of collagen in the graft construct is at least 1.1 g/mL, 7) the construct is selected from the group consisting of a thread, a rope, a ring, a sheet, a tube, and combinations thereof, 8) the construct is woven or braided, 9) the composition further comprises an exogenous population of cells, 10) the exogenous population of cells is selected from the group consisting of progenitor cells, fibroblasts, neural cells, osteoblast cells, endothelial cells, and smooth muscle cells, 11) the graft construct further comprises at least one polysaccharide, 12.) the graft construct further comprises at least one proteoglycan.

In one embodiment, an engineered graft construct is described that comprises aligned collagen fibrils, wherein the construct is anisotropic and the ultimate tensile stress of the graft construct is about 0.5 MPa to about 150 MPa. In accordance with this embodiment, 1) the collagen is crosslinked, 2) the collagen is uncrosslinked., 3) the ultimate tensile stress of the graft construct is about 24 MPa to about 88 MPa, 4) the ultimate tensile stress of the graft construct is about 1 MPa to about 5 MPa, 5) the ultimate tensile stress of the graft construct is about 4 MPa to about 40 MPa, 6) the density of collagen in the graft construct is at least 1.1 g/mL, 7) the construct is selected from the group consisting of a thread, a rope, a ring, a sheet, a tube, and combinations thereof, 8) the construct is woven or braided, 9) the composition further comprises an exogenous population of cells, 10) the exogenous population of cells is selected from the group consisting of progenitor cells, fibroblasts, neural cells, osteoblast cells, endothelial cells, and smooth muscle cells, 11) the graft construct further comprises at least one polysaccharide, and/or 12) the graft construct further comprises at least one proteoglycan.

In one embodiment an engineered graft construct is described that comprises aligned collagen fibrils, wherein the construct is anisotropic and the ultimate tensile strain of said graft construct is about 0.5% to about 30%. In accordance with this embodiment, 1) the collagen is crosslinked, 2) the collagen is uncrosslinked, 3) the ultimate tensile strain percent of the graft construct is about 0.5% to about 10%, 4) the ultimate tensile strain percent of the graft construct is about 1% to about 10%, 5) the ultimate tensile strain percent of the graft construct is about 5% to about 30%, 6) the density of collagen in the graft construct is at least 1.1 g/mL, 7) the construct is selected from the group consisting of a thread, a rope, a ring, a sheet, a tube, and combinations thereof, 8) the construct is woven or braided, 9) the composition further comprises an exogenous population of cells, 10) the exogenous population of cells is selected from the group consisting of progenitor cells, fibroblasts, neural cells, osteoblast cells, endothelial cells, and smooth muscle cells, 11) the graft construct further comprises at least one polysaccharide, and/or 12) the graft construct further comprises at least one proteoglycan.

In one embodiment, an engineered graft construct is described that comprises aligned collagen fibrils, wherein the construct is anisotropic and the elastic or linear modulus of said graft construct is about 50 MPa to about 700 MPa. In accordance with this embodiment, 1) the collagen is crosslinked, 2) the collagen is uncrosslinked, 3) the elastic or linear modulus is about 250 MPa to about 700 MPa, 4) the elastic or linear modulus is about 75 MPa to about 500 MPa. 5) the elastic or linear modulus is about 50 MPa to about 100 MPa, 6) the density of collagen in the graft construct is at least 1.1 g/mL, 7) the construct is selected from the group consisting of a thread, a rope, a ring, a sheet, a tube, and combinations thereof, 8) the construct is woven or braided, 9) the composition further comprises an exogenous population of cells, 10) the exogenous population of cells is selected from the group consisting of progenitor cells, fibroblasts, neural cells, osteoblast cells, endothelial cells, and smooth muscle cells, 11) the graft construct further comprises at least one polysaccharide, and/or 12) the graft construct further comprises at least one proteoglycan.

In another embodiment, a method of preparing a collagen matrix is described. The method comprises the steps of, providing a collagen solution, dispensing said collagen solution into an electrochemical cell, wherein said collagen solution is in contact with at least one electrode, applying an electric field to said collagen solution, wherein the current density is about 0.3 A/m² to about 34 A/m², and generating a pH gradient in the collagen solution, wherein said collagen positions at the isoelectric point of the collagen in said solution.

In various embodiments of the embodiment described in the preceding paragraph, 1) positioning of collagen comprises alignment or accumulation of collagen at the isoelectric point of the collagen in said solution, 2) positioning of collagen comprises gelling of collagen at the isoelectric point of the collagen in said solution, 3) the amount of collagen in said collagen solution is about 0.5 mg/ml to about 6 mg/ml, 4) the electric field has an electric field strength of about 100 V/m to about 30 KV/m, 5) the voltage applied to said electrochemical cell is at least 1.2 V, 6) the at least one electrode is tubular, 7) the at least one electrode comprises two linear electrodes, 8) the electrodes are parallel line electrodes, 9) the at least one electrode is in the form of a ring, 10) the electrodes are in the form of plates, and/or 11) the at least one electrode is formed from carbon, stainless steel, gold, gold-plated metals, platinum, or a combination thereof.

In another embodiment, a kit is described. The kit comprises an engineered graft construct comprising aligned collagen fibrils, wherein the construct is anisotropic and the fibril area fraction of the construct is about 80% to about 100%. In yet another embodiment, the kit comprises an engineered graft construct, wherein the graft construct further comprises at least one polysaccharide. In yet another embodiment, the kit comprises an engineered graft construct, wherein the graft construct further comprises at least one proteoglycan. In accordance with these embodiments, 1) the kit comprises an engineered graft construct that has been disinfected, 2) the kit further comprises a vial of cells.

In another embodiment, an apparatus for aligning collagen molecules is described. The apparatus comprises a substrate, a first electrode and a second electrode each positioned in contact with the substrate, the first electrode and the second electrode having a gap therebetween configured to receive a collagen solution containing a number of collagen molecules, a moisture chamber having the substrate, the first electrode, and the second electrode positioned therein, and a power supply electrically connected to the first electrode and the second electrode, the power supply configured to provide a voltage to the first electrode and the second electrode to create an electric field in the gap such that each collagen molecule of the number of collagen molecules received in the gap is aligned along its respective isolectric point.

In various embodiments of the embodiment described in the preceding paragraph, 1) the apparatus further comprises a resistive element connected to the power supply and one of the first and second electrodes, 2) the substrate is formed of glass, plastic, ceramic, metal, or combinations thereof, 3) the power supply is a dc power supply, 4) the power supply is an ac power supply, 5) the first electrode and the second electrode each comprise a wire, 6) the first electrode and the second electrode are each plate-shaped, 7) the first electrode is tubular-shaped and the second electrode is positioned within the first electrode and extends along the longitudinal axis of the first electrode, 8) the first electrode comprises a loop and the second electrode is positioned within the loop, and/or 9) the first and second electrodes are each formed of a material selected from a group consisting of carbon, stainless steel, gold, gold-plated metals, and platinum.

In another embodiment, a method for aligning collagen contained in a collagen solution along its isoelectric point is described. The method comprises the steps of dispensing the collagen solution having a number of molecules in a gap between a first electrode and a second electrode, applying a voltage to the first and the second electrodes to produce an electric field in the gap, and controlling the voltage applied to the first and the second electrodes to align each collagen molecule of the number of molecules along its respective isoelectric point.

In various embodiments of the embodiment described in the preceding paragraph, 1) the dispensing the collagen solution having a number of collagen molecules in a gap between a first electrode and a second electrode comprises dispensing the collagen solution having a number of collagen molecules in a gap between a first plate-shaped electrode and second plate-shaped electrode, 2) the dispensing the collagen solution having a number of collagen molecules in a gap between a first electrode and a second electrode comprises dispensing the collagen solution having a number of collagen molecules in a gap between a first electrode comprising a loop and a second electrode positioned within the loop, and/or 3) the dispensing the collagen solution having a number of collagen molecules in a gap between a first electrode and a second electrode comprises dispensing the collagen solution having a number of collagen molecules in a gap between a first tubular-shaped electrode and a second tubular-shaped electrode positioned within the first tubular-shaped electrode.

In another embodiment, a nerve guide construct is described that comprises aligned collagen fibrils, wherein the construct is anisotropic. In accordance with this embodiment, 1) the fibril area fraction of the collagen fibrils can be about 80% to about 100%, 2) the construct can be a structure in a form selected from the group consisting of a thread, a rope, a ring, a sheet, a tube, and combinations thereof, 3) the construct can be woven or braided, 4) the construct can further comprise an exogenous population of cells, 5) the exogenous population of cells can be selected from the group consisting of progenitor cells, stem cells, and neural cells, 6) the neural cells can be Schwann cells, and 7) the elastic or linear modulus of the collagen fibrils can be about 50 MPa to about 100 MPa.

### BRIEF DESCRIPTION OF THE DRAWINGS

FIG. 1. shows polarized optical images of collagen rope by using a first order wavelength plate: Panel A) parallel to the slow axis of a gypsum plate; Panel B) perpendicular to the slow axis of a gypsum plate (Bar=100 um).

FIG. 2. shows images of collagen gel formation under an electric field by using a pH indicator order wavelength plate: Panel A) pH induced color change of a collagen gel between two electrodes (image is blown vertically to amplify the variation in color tone between wire electrodes); Panel B) aligned collagen gel perpendicular to the slow axis of a gypsum plate.

FIG. 3. shows confocal images of an anisotropic collagen gel showing the orientation of collagen bundles (Panel A, bar=50 um) and SEM images of an anisotropic collagen gel that is crosslinked (Panel B, bar=4 um).

FIG. 4. shows the ultimate strength (Panel A) and energy to fracture (Panel B) of collagen gels: 1) isotropic-uncrosslinked (not solid enough to be tested); 2) anisotropic-uncrosslinked; 3) isotropic-crosslinked; and 4) anisotropic-crosslinked [(N=4 for groups 3 and 4)]. The dots on the bars in A are outliers which were not included in calculation of mean and standard deviation.

FIG. 5. shows the experimental setup for the electrochemically induced collagen assembly process for production of aligned collagen fiber bundles.

FIG. 6. shows raman spectra of aligned collagen samples compared with tendon and random collagen gels: Panel A) random collagen gel; Panel B) native tendon bundle; Panel C) bleached tendon fiber; Panel D) aligned collagen fiber (A-D are noncrosslinked). The major peaks of collagen are identified ν (stretch mode) and δ (bending mode).

FIG. 7. shows the pH-gradient between parallel electrodes and a schematic representation of collagen alignment and the congregation mechanism: Panel A) universal pH indicator dye revealed the pH-gradient in the collagen solution between the anode (acidic) and the cathode (basic); Panel B) auto-fluorescent confocal image showing that all of the collagen in the solution was congregated along a fixed band via isoelectric focusing. The aligned collagen band was highly birefringent. (inset, bar=200 μm); Panel C) schematic representation of the electrochemical assembly of collagen molecules. 1=Rotatory alignment, the pH-gradient and electrostatic interactions rotate the molecules normal to the electric field; 2=Isoelectric focusing, charged collagen molecules move towards the isoelectric point where they become uncharged and their mobility is arrested (in actual events, stage 1 and stage 2 are likely to happen simultaneously); 3=Assembly, the high concentration of collagen due to congregation at the isoelectric point and the basic pH between the cathode and isoelectric point facilitate fibrillar assembly of molecules into a macroscale bundle. Depending on the electrodes configuration, collagen can be formed into straight fibers or rings (tubes).

FIG. 8. Time-elapsed compensated polarized optical images of a collagen bundle and a ring formed under electrochemical effects. Panels A-D show that the collagen band structure grows with time at a location between the electrodes. Molecules which are oriented parallel and perpendicular to the slow axis of the gypsum plate (white double arrow) appear blue and yellow, respectively. Note that the entrapped air bubbles (intentionally introduced inside the collagen solution by mixing) changed from circular to elliptical due to lateral compressive forces. This compression force is a result of molecular congregation during isoelectric focusing and improves the orientation and packing density of collagen molecules. Panel E shows a compensated polarized optical image of a collagen ring with molecules aligned circumferentially as evidenced by blue and yellow gradations about the circumference in different orthogonal quadrants. Panel F shows a polarized image showing that the collagen ring is highly birefringent.

FIG. 9. shows the pH-gradient in the collagen solution between two parallel electrodes as revealed by the universal indicator dye (Panel A); a compensated polarized optical microscopic image showing that the collagen in the solution was congregated and assembled along a fixed band via isoelectric focusing [blue color indicates that molecules are oriented parallel to the slow axis (double arrow) of the gypsum plate which is set parallel to the electrodes] (Panel B); a polarized optical image of B demonstrating the rotary alignment of collagen molecules in the region bounded by the band and cathode as indicated by a relatively weaker birefringence [the same region in B is devoid of blue color, therefore, rotational alignment is incomplete and in progress] (Panel C); the resulting collagen band in the solid phase is highly birefringent [inserted bottom image showed that the pH of stained collagen fiber was around 9] (Panel D); an SEM image of a recovered aligned, crosslinked collagen bundle split along its longer axis intentionally to reveal the degree of packing and the uniformity of orientation along the longer axis of the bundle (Panel E); a compensated polarized optical image of a collagen ring with molecules aligned circumferentially as evidenced by blue and yellow graduations about the circumference in different orthogonal quadrants (Panel F).

FIG. 10. shows a comparative analysis of the orientational anisotropy between tendon and the electrochemically aligned collagen bundles via compensated polarized optical microscopy, small angle X-ray scattering (SAXS) and second harmonic generation—nonlinear optical ellipsometry (SHG-NOE) analysis. Panel A) when the bundles were parallel to the direction of slow axis (white double headed arrow) of the gypsum plate in polarized optical microscope both the bleached tendon and oriented collagen construct showed the blue retardation color, indicating the molecules were oriented parallel to the slow axis. Panel B) when the bundles were rotated perpendicular to the slow axis of the gypsum plate, both the bleached tendon and oriented collagen construct changed to the yellow retardation color, confirming the orientation of molecules to be along the longer axes of the bundle. Panel C) shows a SAXS pattern of the tendon. Panel D) shows a SAXS pattern of the aligned, crosslinked collagen bundle. Panel E) shows intensity versus peak position of X ray scattering. The meridonial scattering pattern is affected mostly by the periodic axial electron density which gives rise to diffraction peaks in the SAXS patterns. The positions of q peaks correspond to the period length D of the axial electron density D=n·2π/qₚₑₐₖ, where n is the order of the peak. Panel F) shows SHG-NOE analysis of different groups of fibers. This measurement indicates that the orientational order of the aligned, crosslinked collagen construct and native tendon fibers differed significantly from the random, crosslinked collagen gel. Collagen fibrils in both tendon and aligned collagen construct are aligned along the long axis of the bundle. “*” denotes significant difference between this group and other groups (N=27, p<0.05).

FIG. 11. shows SEM and TEM images of aligned and random collagen constructs. Panel A) SEM image of a collagen gel prepared without employing the electrochemical process. The fabric displays a random network of collagen fibers. Panel B) TEM of the random collagen shows the typical periodic D-banding in fibers. Panel C) SEM image shows that aligned collagen fiber prepared via the electrochemical alignment method is composed of uniformly oriented small fibers. Panel D) TEM of fibers teased from the aligned collagen construct. Electrochemically synthesized fibers are 25-40 nm in diameter. Each fiber is composed of fibrils of about 8 nm in diameter. The d periodic banding is not discernable from TEM images; however, its presence was discerned from the SAXS pattern.

FIG. 12. shows the mechanical properties of collagen constructs prepared by different methods: Panel A) ultimate tensile strength; Panel B) tensile modulus (N=10). “*” denotes significant difference between the connected group and subgroup.

FIG. 13. shows confocal fluorescence micrographs (red=Alexafluor 488 labeled actin in cells, green=collagen reflection) showing cell attachment on the aligned collagen construct within 24 hours: Panel A) overlay image of collagen and attached cells; Panel B) fluorescence image of the cells alone.

FIG. 14. shows four basic electrode configurations (wire, plate, ring, tube) that can be used in the electrochemical process described to produce: Panel A) fiber bundles; Panel B) sheets; Panel C) rings; and Panel D) tubes of collagen.

FIG. 15. shows a rat tendon-derived fibroblast cell migration assay and universal standard cell direct contact test on collagen fibers. Panel A) shows a chematic drawing and the micrograph of the cell migration construct. Panel B) shows fluorescent micrographs demonstrating the 6-day migration of fibroblasts labeled with Alexa Fluor 488 phalloidin on the aligned, crosslinked and random, crosslinked collagen fibers. Panel C) shows confocal fluorescence image of cytoskeletal actin filaments oriented along the aligned, crosslinked bundle (top inset is higher magnification of the confocal image and the bottom inset is histological image of cells stained with hematoxylin and eosin). Panel D) shows actin filament of cells on random, crosslinked collagen fibers. Panel E) shows the migration distance of fibroblasts on the 3rd and the 6th days. The measured migration distances are significantly different from each other (n=3, p<0.05, * indicates statistically significant difference), indicating that the cells migrate a greater distance on the aligned collagen fibers compared to the random collagen fibers. Panel F) shows direct contact test for assessment of the biocompatibility of aligned, crosslinked collagen fibers. The viable cells are quantified with a CellTiter 96 AQ cell proliferation assay. There is no statistically significant difference between the experimental group and the control group (n=4, p>0.05) indicating that the aligned, crosslinked collagen fibers are not cytotoxic.

FIG. 16. shows the mechanical properties of collagen constructs prepared by different methods. Panel A) shows typical stress-strain curve of aligned, crosslinked collagen and native tendon showing toe, heel, and linear region. Panel B) shows ultimate tensile stress. Panel C) shows tensile modulus (N=10/group).

FIG. 17. shows in vitro degradation of aligned, crosslinked collagen fibers in the presence of bacterial collagenase type I. The weights of the remaining collagen bundles at different time-points are shown (3 samples per time point).

FIG. 18. shows a demonstration of aligned, crosslinked collagen fiber bundles for tendon/ligament replacement. Panel A) shows tendon-derived fibroblast cells migration and proliferation after 7 days on a construct prepared by twisting 4 collagen fiber bundles. Panel B) shows a schematic representation of application of aligned, crosslinked collagen bundles for tendon/ligament replacement. Multiple bundles can be grouped together by braiding (top) or parallel binding to form a synthetic collagen construct with desired dimensions.

FIG. 19. shows a phase contrast image of tendon-derived cells showing typical fibroblast-like morphology. Cells between passages 3 and 5 were used for the migration study and the USP direct contact test.

FIG. 20. shows an optical image of mineralized aligned collagen fiber. Panels A and B) show an optical image of a mineralized aligned collagen fiber without crosslinking at different magnifications; Panel C) shows an optical image of aligned collagen bundles.

FIG. 21. shows a dialyzed monomeric collagen used as the stock for the electrochemical process. The turbidity test indicates that the stock solution does not form a gel at room temperature (Generally, gelation occurs after the addition of PBS and adjusting the temperature to 37° C.).

FIG. 22. shows an image of a Sirius red stained individual aligned collagen bundle showing the dimensions (about 7 cm length and 400 mm diameter).

FIG. 23. shows SAXS patterns of the freshly aligned collagen (Panel A), PBS treated aligned collagen (Panel B), aligned-CX collagen (Panel C), and the natural tendon (Panel D).

FIG. 24. shows intensity versus peak position of X-ray scattering.

FIG. 25. shows histological sections taken transversely to the longitudinal axis of a 3D construct composed of 3 bundles (sandwiched between two PTFE sheets to aid in histological processing). The micrograph shows that the cells can migrate and populate the space in between the bundles ( [symbol] ) as well as the outer surface of bundles ( [symbol] ).

FIG. 26. shows neural cells cultured on an anisotropic collagen construct, prepared as herein described.

FIG. 27. shows neural cells cultured on randomly oriented collagen.

### DETAILED DESCRIPTION OF THE INVENTION

While the invention is susceptible to various modifications and alternative forms, illustrative embodiments are described herein. It should be understood, however, that there is no intent to limit the invention to the particular forms described, but on the contrary, the intention is to cover all modifications, equivalents, and alternatives falling within the spirit and scope of the invention.

In one embodiment, an engineered graft construct is provided comprising aligned collagen fibrils wherein the construct is anisotropic, and wherein the fibril area fraction is about 80% to about 100%. In another embodiment, an engineered graft construct is provided comprising aligned collagen fibrils wherein the construct is anisotropic, and wherein the ultimate tensile stress of the graft construct is about 0.5 MPa to about 150 MPa. In yet another embodiment, an engineered graft construct is provided comprising aligned collagen fibrils wherein the construct is anisotropic, and wherein the ultimate tensile strain of the graft construct is about 0.5% to about 30%. In another illustrative aspect, an engineered graft construct is provided comprising aligned collagen fibrils wherein the construct is anisotropic, and wherein the elastic or linear modulus of said graft construct is about 40 MPa to about 700 MPa.

Illustratively, in another aspect, a method of preparing a collagen matrix is provided. The method comprises the steps of providing a collagen solution, dispensing the collagen solution into an electrochemical cell, wherein the collagen solution is in contact with at least one electrode, applying an electric field to the collagen solution, wherein the current density is about 0.3 A/m² to about 34 A/m², and generating a pH gradient in the collagen solution, wherein the collagen positions at the isoelectric point of the collagen in the solution. In another method embodiment, a method for aligning collagen contained in a collagen solution along its isoelectric point is provided. The method comprises the steps of dispensing the collagen solution having a number of molecules in a gap between a first electrode and a second electrode, applying a voltage to the first and the second electrodes to produce an electric field in the gap, and controlling the voltage applied to the first and the second electrodes to align each collagen molecule of the number of molecules along its respective isoelectric point.

Apparatus embodiments for performing the method described herein are also provided. In one illustrative aspect, an apparatus for aligning collagen molecules is provided. The apparatus comprises a substrate, a first electrode and a second electrode each positioned in contact with the substrate, the first electrode and the second electrode having a gap therebetween configured to receive a collagen solution containing a number of collagen molecules, a moisture chamber having the substrate, the first electrode, and the second electrode positioned therein, and a power supply electrically connected to the first electrode and the second electrode, the power supply configured to provide a voltage to the first electrode and the second electrode to create an electric field in the gap such that each collagen molecule of the number of collagen molecules received in the gap is aligned along its respective isoelectric point.

As used herein, a “modulus” can be an elastic or linear modulus (defined by the slope of the linear region of the stress-strain curve obtained using conventional mechanical testing protocols; i.e., stiffness), a compressive modulus, or a shear storage modulus.

As used herein, a “fibril area fraction” is defined as the percent area of the total area occupied by fibrils in a cross-sectional surface of the matrix; 2-dimensional) or a fibril volume fraction (the percent area of the total area occupied by fibrils in 3 dimensions).

As used herein, tensile or compressive stress “a” is the force carried per unit of area and is expressed by the equation:

$$σ = \frac{P}{A} = \frac{P}{a b}$$

where:
s=stress
P=force
A=cross-sectional area
a=width
h=height

The force (P) produces stresses normal (i.e., perpendicular) to the cross section of the part (e.g., if the stress tends to lengthen the part, it is called tensile stress, and if the stress tends to shorten the part, it is called compressive stress).

As used herein, “tensile strain” is the strain caused by bending and/or stretching a material.

As used herein, “anisotropic” means load-bearing in a dominant direction due to alignment of collagen fibrils.

In one illustrative embodiment, a method is described for preparing an engineered graft construct. In one illustrative embodiment, the method comprising the steps of, providing a collagen solution, dispensing the collagen solution into an electrochemical cell wherein said collagen solution is in contact with at least one electrode, applying an electric field to said collagen solution, wherein the current density is about 0.3 A/m² to about 34 A/m², and generating a pH gradient in the collagen solution wherein the collagen positions at the isoelectric point of the collagen in said solution. In another embodiment, the collagen aligns or accumulates at the isoelectric point of the collagen in said solution. In yet another embodiment, the collagen undergoes fibrillogenesis at the isoelectric point of the collagen in the solution. Fibrillogenesis of the collagen in the solution results in collagen gelling at the isoelectric point of the collagen in the solution.

In one embodiment, the current density is about 0.3 to about 34 A/m². In various illustrative embodiments, the current density is about 0.3 to about 11 A/m², about 0.4 to about 11 A/m², about 0.45 to about 9.5 A/m², about 0.45 to about 6 A/m², about 0.45 to about 7.5 A/m², about 0.45 to about 8.5 A/m², about 0.5 to about 9.5 A/m², about 0.5 to about 8.5 A/m², about 1 to about 9 A/m², about 1 to about 34 A/m², and 10 to about 34 A/m²

In one illustrative aspect, aligned collagen constructs with high packing density can be formed by using a weak electric field as described herein. In one embodiment, crosslinking of the engineered graft constructs allows the constructs to retain orientation and improves the strength of the construct. The engineered graft constructs as described herein show improved mechanical strength and toughness compared to random collagen gels. The unique electrochemical process described herein can involve a variety of processes, including but not limited to, molecular alignment due to pH-gradient and electrostatic effects, molecular congregation via isoelectric focusing, and pH-induced gelation of molecules near the isoelectric point. The use of a low level electric field and current allow for sufficient time for alignment perpendicular to the electric field, migration, congregation, and gelation to take place.

These conditions result in formation of an aligned collagen construct with desired compositional, microstructural, and mechanical characteristics. Illustratively, these compositional, microstructural, and mechanical characteristics can include fibril length, fibril diameter, number of fibril-fibril connections (e.g., cross-links), fibril density, fibril organization, matrix composition, 3-dimensional shape or form, and viscoelastic, tensile, shear, or compressive behavior (e.g., failure stress, failure strain, and modulus), permeability, degradation rate, swelling, hydration properties (e.g., rate and swelling), and in vivo tissue remodeling and bulking properties, viscosity of interstitial fluid and desired in vitro and in vivo cell responses. The engineered graft constructs described herein have desirable biocompatibility and in vitro and in vivo remodeling properties, among other desirable properties.

In one embodiment, the engineered graft construct may be uncrosslinked. In another embodiment, the graft construct may be crosslinked. In various illustrative embodiments, crosslinking agents, such as carbodiimides, aldehydes, lysl-oxidase, N-hydroxysuccinimide esters, imidoesters, hydrazides, and maleimides, as well as various natural crosslinking agents, including genipin, and the like can be added before, during, or after polymerization of the collagen in solution.

As used herein “sterilization” or “sterilize” or “sterilized” means removing unwanted contaminants including, but not limited to, endotoxins, nucleic acid contaminants, and infectious agents.

In various illustrative embodiments, the engineered collagen construct can be disinfected and/or sterilized using conventional sterilization techniques including glutaraldehyde tanning, formaldehyde tanning at acidic pH, propylene oxide or ethylene oxide treatment, gas plasma sterilization, gamma radiation, electron beam, and/or peracetic acid sterilization. Sterilization techniques which do not adversely affect the structure and biotropic properties of the construct can be used. Illustrative sterilization techniques are exposing the engineered collagen construct, to peracetic acid, 1-4 Mrads gamma irradiation (or 1-2.5 Mrads of gamma irradiation), ethylene oxide treatment, or gas plasma sterilization. In one embodiment, the engineered graft construct can be subjected to one or more sterilization processes. In illustrative embodiments, the collagen in solution can also be sterilized.

In one illustrative embodiment, the collagen solution provided can have a collagen concentration ranging from about 0.5 mg/ml to about 6 mg/ml. In various embodiments, the collagen concentration may range from about 0.5 mg/ml to about 10 mg/ml, about 0.1 mg/ml to about 6 mg/ml, about 0.1 mg/ml to about 5 mg/ml, about 0.5 mg/ml to about 2 mg/ml, about 0.5 mg/ml to about 3 mg/ml, about 0.5 mg/ml to about 4 mg/ml, about 0.5 mg/ml to about 5 mg/ml, about 1 mg/ml to about 6 mg/ml, about 1 mg/ml to about 5 mg/ml, about 1 mg/ml to about 4 mg/ml, about 1 mg/ml to about 3 mg/ml, about 2 mg/ml to about 5 mg/ml, and about 2 mg/ml to about 4 mg/ml.

In various illustrative embodiments, the collagen used herein may be any type of collagen, including collagen types Ito XXVIII, alone or in any combination. In various aspects, the isolated collagen can also contain endogenous or exogenously added non-collagenous proteins (e.g., fibronectin, fibrinogen, keratin or silk proteins), glycoproteins, proteoglycans, polysaccharides, glycosaminoglycans (e.g., chondroitins and heparins), or the like. The engineered graft constructs prepared by the methods described herein can serve as constructs for the regrowth of endogenous tissues at the implantation site (e.g., biological remodeling) which can assume the characterizing features of the tissue(s) with which they are associated at the site of implantation, insertion, or injection.

In any of these embodiments the engineered graft construct may further comprise an added population of cells. The added population of cells may comprise one or more cell populations. In various embodiments, the cell populations comprise a population of non-keratinized or keratinized epithelial cells or a population of mesodermally derived cells selected from the group consisting of endothelial cells, neural cells, osteoblast cells, fibroblasts, smooth muscle cells, skeletal muscle cells, cardiac muscle cells, multi-potential progenitor cells (e.g., stem cells, including bone marrow progenitor cells), and osteogenic cells. In various embodiments, the collagen construct can be seeded with one or more cell types in combination.

In various illustrative embodiments, qualitative and quantitative microstructural characteristics of the engineered matrices can be determined by scanning electron microscopy, transmission electron microscopy, confocal microscopy, second harmonic generation multi-photon microscopy. In another embodiment, tensile, compressive and viscoelastic properties can be determined by rheometry or tensile testing. All of these methods are known in the art or are further described in the Examples section of this application.

In various illustrative embodiments, the alignment of the isolated collagen is conducted at a pH selected from the range of about 5.0 to about 11.0, and in one embodiment alignment is conducted at a pH selected from the range of about 6.0 to about 9.0, and in one embodiment alignment is conducted at a pH selected from the range of about 3.0 to about 10.0, and in another embodiment the alignment of the isolated collagen is conducted at a pH selected from the range of about 7.0 to about 8.5, and in another embodiment the alignment of the isolated collagen is conducted at a pH selected from the range of about 7.3 to about 7.4.

In various aspects, the collagen constructs of the present invention can be combined with nutrients, including minerals, amino acids, sugars, peptides, proteins, vitamins (such as ascorbic acid), or glycoproteins that facilitate cellular proliferation, such as laminin and fibronectin, hyaluronic acid, or growth factors such as epidermal growth factor, platelet-derived growth factor, transforming growth factor beta, or fibroblast growth factor, and glucocorticoids such as dexamethasone. In other illustrative embodiments, cross-linking agents, such as carbodiimides, aldehydes, lysl-oxidase, N-hydroxysuccinimide esters, imidoesters, hydrazides, and maleimides, as well as natural crosslinking agents, including genipin, and the like can be added before the addition of cells.

In another illustrative embodiment, the engineered graft constructs contain fibrils with specific characteristics, including, but not limited to, a fibril area fraction of about 80% to about 100%, about 85% to about 100%, about 90% to about 100%, about 80% to about 90%, about 80% to about 94%, about 80% to about 95%, about 80% to about 97%, about 90% to about 94%, about 90% to about 95%, or about 90% to about 98%.

In yet another embodiment, the engineered graft constructs have a modulus of about 50 MPa to about 1.5 GPa, about 50 MPa to about 500 MPa, about 100 MPa to 500 MPa, about 50 MPa to about 1000 MPa, about 50 MPa to about 700 MPa, about 100 MPa to about 700 MPa, about 100 MPa to 600 MPa, about 150 MPa to about 500 MPa, or about 200 MPa to about 700 MPa. In other illustrative embodiments, the anisotropic collagen constructs can have an elastic or linear modulus of about 50 MPa to about 675 MPa, about 50 MPa to about 675 MPa, about 250 MPa to about 700 MPa, about 75 MPa to about 500 MPa, and about 50 MPa to about 100 MPa.

In one embodiment, the collagen constructs have a tensile stress of about 0.5 MPa to about 150 MPa, about 0.5 MPa to about 5 MPa, about 0.5 MPa to about 1 MPa, about 0.5 MPa to about 2 MPa, about 1 MPa to about 4 MPa, about 1 MPa to about 5 MPa, about 0.5 MPa to about 100 MPa, about 1 MPa to about 100 MPa, about 1 MPa to about 95 MPa, about 1 MPa to about 90 MPa, about 1 MPa to about 88 MPa, about 1 MPa to about 80 MPa, about 1 MPa to about 75 MPa, about 2 MPa to about 90 MPa, about 4 MPa to about 90 MPa, about 4 MPa to about 75 MPa, about 4 MPa to about 50 MPa, about 4 MPa to about 40 MPa, about 24 MPa to about 88 MPa, about 25 MPa to about 50 MPa, about 25 MPa to about 75 MPa, and about 25 MPa to about 100 MPa.

In one embodiment, the collagen constructs have a tensile strain of about 0.5% to about 30%, about 0.5% to about 20%, about 1% to about 30%, about 0.5% to about 10%, about 0.5% to about 5%, about 1% to about 18%, about 10% to about 24%, about 1% to about 24%, about 5% to about 25%, about 7% to about 24%, about 4% to about 16%, and about 1.2% to about 6%.

In another embodiment, the density of collagen in the engineered construct is at least 1.10 g/mL. For example, in various illustrative embodiments, the density of collagen in the construct may be about 1.12 g/mL, about 1.14 g/mL, about 1.15 g/mL, about 1.16 g/mL, about 1.18 g/mL, about 1.20 g/mL, about 1.22 g/mL, about 1.25 g/mL, about 1.28 g/mL, about 1.3 g/mL, about 1.35 g/mL, about 1.40 g/mL, about 1.45 g/mL, about 1.50 g/mL, about 1.75 g/mL, about 2.00 g/mL, about 2.20 g/mL, about 2.50 g/mL, and about 3.00 g/mL or greater.

As discussed above, in accordance with one embodiment, cells can be added to the engineered constructs after polymerization of the engineered constructs. The engineered constructs comprising the cells can be subsequently injected or implanted in a host for use as a graft construct. In another embodiment, the cells on or within the engineered constructs can be cultured in vitro, for a predetermined length of time, to increase the cell number or to induce desired remodeling prior to implantation or injection into a host. In a further embodiment, the cells can be cultured in vitro, for a predetermined length of time, to increase cell number and the cells can be separated from the construct and implanted or injected into the host in the absence of the construct.

In accordance with one embodiment, a kit is provided comprising the engineered graft construct. In one embodiment, cells may constitute a component of the kit. In accordance with one embodiment, the kit comprises a graft construct comprising aligned collagen fibrils wherein the construct is anisotropic, and wherein the fibril area fraction is about 80% to about 100%. However, the fibril area fraction may vary as herein described. In various illustrative embodiments, the engineered construct in the kit may comprise various other components, including non-collagenous proteins, polysaccharides and proteoglycans. In one embodiment, the kit comprises separate vessels, each containing one of the following components: engineered graft construct, a disinfecting agent, and non-collagenous proteins, polysaccharides, or proteoglycans. The kits can further comprise instructional materials describing methods for using the kit reagents or describing methods for using preformed, aligned collagen constructs.

In yet another embodiment, the kit further comprises a vial of cells, including but not limited to, a population of non-keratinized or keratinized epithelial cells or a population of mesodermally derived cells selected from the group consisting of endothelial cells, neural cells (e.g., Schwann cells), osteoblast cells, fibroblasts, smooth muscle cells, skeletal muscle cells, cardiac muscle cells, multi-potential progenitor cells (e.g., stem cells, including bone marrow progenitor cells), pericytes, and osteogenic cells. In various illustrative embodiments, the kit comprises one or more vials of cells and may comprise one or more cell types and cell culture reagents.

As used herein, the electric field between one or more electrodes is about 100 V/m to about 30 KV/m. In one embodiment, the electric field between one or more electrodes is about 1000 to about 2000 V/m (i.e., about 1 to about 2 KV/m). In various illustrative embodiments, the electric field is about 100 V/m to about 25 kV/m, about 170 V/m to about 25 kV/m, about 200 V/m to about 25 kV/m, about 300 V/m to about 25 kV/m, about 500 V/m to about 1500 V/m, about 1000 V/m to about 1500 V/m, about 1000 V/m to about 2500 V/m, about 1200 V/m to about 2000 V/m, about 1200 V/m to about 1800 V/m, and about 500 V/m to about 2500 V/m.

In one embodiment, the voltage of the power supply is about 6V to about 30 V. In another embodiment, the actual voltage between the electrodes is at least 1.20 V. Illustratively, any voltage that allows for the electrolysis of water and pH gradient formation may be useful in the method described herein.

The power supply as used herein may be any power supply, including ac or dc power supplies. In another illustrative embodiment, the current is about 3.9 μA to about 90 μA. In one embodiment, the current applied to the collagen solution is about 1 μA to about 10 μA is applied to said collagen solution. In another embodiment, the current applied to the collagen solution is about 1 μA to about 9 μA, about 0.5 μA to about 11 μA, about 1 μA to about 8 μA, about 1 μA to about 7 μA, about 1 μA to about 5 μA, about 2 μA to about 8, and about 2 μA to about 10 μA. In another illustrative embodiment the current applied to the collagen solution is about 1 μA to about 400 μA, about 0.5 μA to about 90 μA, about 0.5 μA to about 50 μA, and about 1 μA to about 90 μA. As the size of the cell changes, the current value will change. Therefore, any current that produces a current density as herein described can be used.

In one embodiment, the at least one electrode may comprise any inert material, including but not limited to, carbon, stainless steel (e.g., 316 stainless steel, etc.), gold, gold-plated metals, platinum, or any other art recognized material useful for generating an electric field. In another embodiment, the at least one electrode comprises two or more electrodes in contact with the collagen solution, wherein the distance between the electrodes is about 0.5 mm to about 5 mm. However, distances between the electrodes that are greater that 5 mm will also be useful in the method described herein.

In various illustrative embodiments, the at least one electrode may be configured in the form of a tube, a line, a ring, or a plate. In yet another embodiment, the at least one electrode comprises two electrodes, wherein the electrodes are parallel line electrodes. Different electrode configurations generate a variety of geometries, including tubular and sheet-like geometries, which could be utilized for vascular repair, nerve regeneration, and skin replacement. The various embodiments, as herein described, result in formation of anisotropic collagen fiber bundles (e.g., a thread or rope), anisotropic collagen sheets, anisotropic collagen rings, and anisotropic collagen tubes. In one aspect, the anisotropic collagen tubes made by the process described herein can be used for tissue engineering applications, such as small diameter blood vessels or nerve guide tubes. As used herein an aligned collagen “thread” is a single strand of collagen. As used herein an aligned collagen “rope” is a twisted or braided strand of one or more collagen fibers useful in accordance with the invention described herein.

In another illustrative embodiment, the aligned collagen construct can be mineralized, for example, with a calcium phosphate, such as hydroxyapatite, and the like. The mineralized aligned collagen constructs as described herein may be used, for example, for hard tissue repair. In various illustrative embodiments, the mineralized aligned collagen constructs may be used for hard tissue repair with or without the addition of cells, for example, osteoblast cells.

The following examples illustrate specific embodiments in further detail. These examples are provided for illustrative purposes only and should not be construed as limiting the invention or the inventive concept in any way.

### EXAMPLES

### Example 1

### Electrochemical Alignment of Collagen

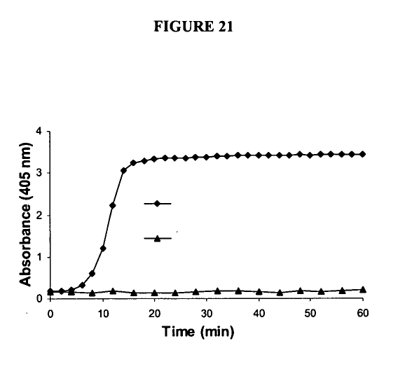

Ten mL of type-I collagen (6 mg/mL 97% bovine hide, INAMED Corporation, Santa Barbara, Calif.) was dialyzed (MW_{cut off}=3.5 kDa) against ultrapure water at 5° C. for 72 hours to remove salts. The dialyzed collagen had the characteristics of normal acidic soluble monomeric collagen and did not undergo fibrillogenesis before the onset of the electrochemical process which took place at room temperature (FIG. 21). The dialyzed collagen underwent fibril formation only after the addition of 10× phosphate buffered saline (PBS) and adjustment of the temperature to 37° C. at pH 7.4 (FIG. 21). This indicates that the dialyzed collagen mainly existed in molecular form instead of fibrillar form at the onset of electric current application.

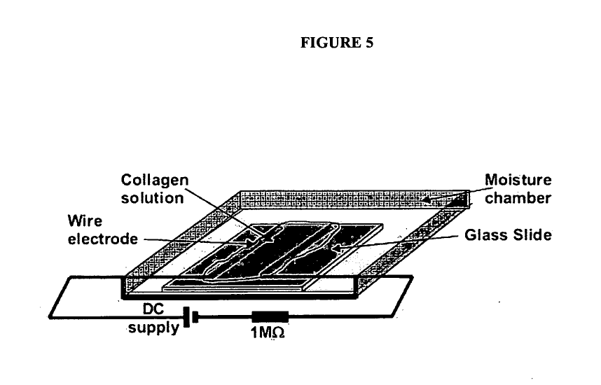

Two stainless steel electrode wires (0.003″ diameter, Sigmund Cohen Corporation, New York) were stripped of their insulating sheet along an inch-long segment and positioned parallel on a glass slide with separation (e.g., 0.5 mm to 5 mm). The gap between the electrodes was filled with the collagen solution (≧6 mg/ml collagen in solution). The dialyzed collagen solution was a transparent viscous fluid. The solution-loaded glass slide (with electrodes) was placed in a humidity chamber (e.g., a Petri dish with the bottom lined with a gauze pad soaked in PBS to maintain humidity). The electrodes were connected to the DC voltage source and a 1 MΩ resistor in series (see FIG. 5). The electric field between two electrodes was about 100 to about 200 V/m. The electrical current across the electrodes was maintained in the 3-5 μA range by connecting the resistor in series with the solution and the DC power supply (FIG. 5). At a supply voltage of 6 V, the current through the collagen solution was measured as 3.5 μA (the actual voltage between two electrodes may be higher than 1.23 V to allow electrolysis of water and pH gradient formation, thus, allowing formation of the aligned collagen construct). The entire assembly was placed on the rotating stage of an optical microscope (Olympus BX 51) equipped with a polarizer, a gypsum plate, and an analyzer. Molecules appear blue when they are rotated parallel to the slow axis of the gypsum plate and yellow when oriented perpendicular to the slow axis of the plate. A camera connected to the microscope recorded the formation of the birefringent band continuously for 1 hour. A current density of about 0.45 to about 9.5 A/m² can be used in the procedure described herein.

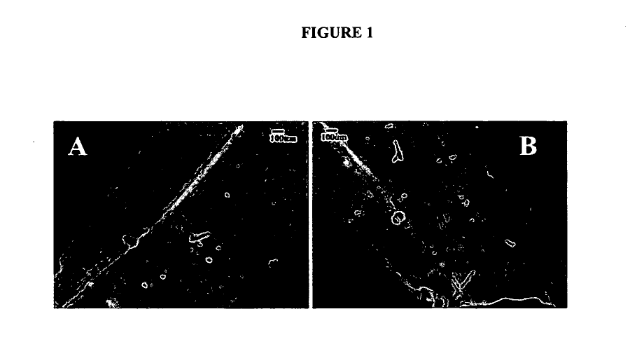

A band was observed to form close and parallel to the cathode within minutes of application of the field. Dimensions of the band were several hundreds of microns in width and about an inch in length. The band progressively grew in width during the course of hours. Accompanied with band formation was a visible reduction in the viscosity of the solution everywhere in the gel, except the band location, suggesting a phoretic mobility of collagen molecules towards the cathode. The uniformity of the orientation of molecules within the band was confirmed by a blue appearance when the band was oriented along the slow axis of the gypsum plate and yellow when band was rotated perpendicular to the plate (FIG. 1, Panels A and B). After the confirmation of the congregation and alignment of molecules along a band by polarized imaging, the current was interrupted, PBS was added and the sample was collected with a pair of tweezers. The freshly aligned collagen bundles were incubated in 10×PBS solution (pH 7.4, 37° C.) for 12 h prior to crosslinking. The average dimension of bundles varied in the range of 50-400 μm diameter and 3-7 cm length depending on the length of electrodes. The aligned collagen bundles were crosslinked in 15 ml of 0.625% genipin (Wako Pure Chemical, Osaka, Japan) in a sterile 1×_PBS solution at 37° C. for 3 days.

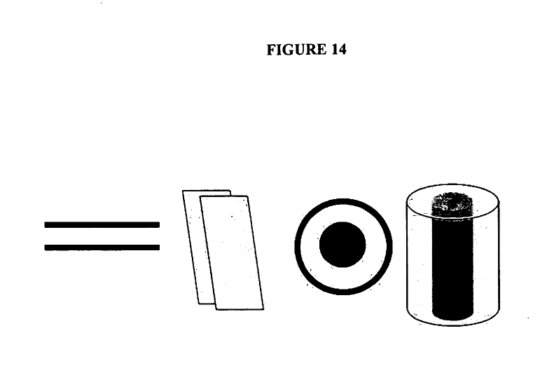

A similar experimental procedure was followed for collagen ring formation, with the exception that the cathode was wound in a ring conformation and the tip of another wire was positioned at the center of the circle to act as the anodic point-source (see Example 3). The current between the anode and the cathode for the ring experiment was 4.1 μA at the supply voltage of 6V. Similar experiments were also conducted for collagen fiber bundle formation (FIG. 14, Panel A), collagen sheet formation (FIG. 14, Panel B), and collagen tube formation (FIG. 14, Panel D).

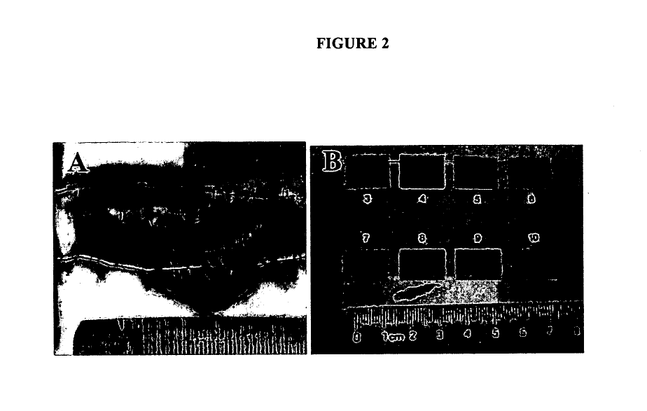

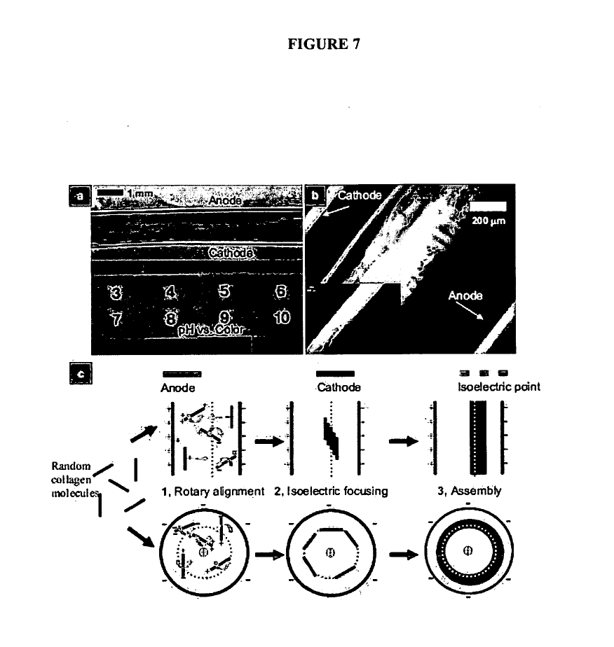

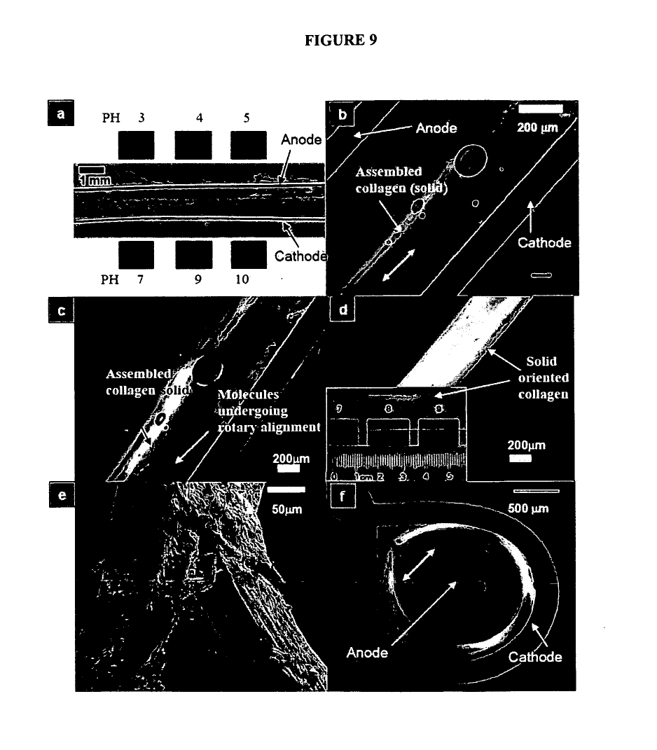

Electrochemical reactions in the solution (e.g., electrolysis of water) generated a pH-gradient between the electrodes, as confirmed by the addition of a universal pH indicator dye (Universal indicator solution pH 3.0-10.0, Sigma) to the solution. The collagen solution near the anode acquires an acidic pH whereas the solution near the cathode becomes basic (FIG. 7, Panel A and FIG. 9, Panel A). To demonstrate the role of the pH gradient in the electrochemical alignment process, a similar experiment was set up as shown in FIG. 5, except that the collagen solution was not in contact with the electrode. Since there is no electrochemical reaction in the collagen solution there is no pH gradient. The collagen solution did not change in orientation and remained isotropic at the applied voltage of 6V. Band formation coincided with an approximate pH of 8 (FIG. 2, Panels A and B).

Charged functional groups are uniformly distributed along the length of the collagen molecule (˜300 nm), where the charge is pH dependent. As an ampholytic molecule, the net charge of collagen molecules in the solution undergoes a transition from negative to positive as the pH decreases from the cathode towards the anode. In response to the external electric field, collagen molecules migrate toward the cathode or anode through the pH gradient until reaching their isoelectric point, where they have no net charge. This results in the entrapment of all collagen molecules at the isoelectric point, as viewed by autofluorescence confocal imaging (FIG. 7, Panel B). Once the congregation and self-assembly of aligned collagen molecules is complete in the vicinity of the isoelectric point, the resulting collagen band assumes a densely packed crystal-like conformation as indicated by the strong birefringence (FIG. 7, Panel B, insert).

The electrochemical set-up to form aligned collagen bundles involved two wire electrodes (0.254 mm diameter, 25 mm length, FIG. 9, Panels A-C and FIG. 5) which were mounted parallel to each other on a glass slide. The gap between the two electrodes was filled with a salt-free collagen solution. The electric current and the voltage across the electrodes were adjusted at about 3.5 μA and 2.5 V DC, respectively, by connecting a resistor in series with the solution (FIG. 5). The current density flowing across the collagen solution (current/height of collagen solution/length of electrodes) was about 0.55 A/m2 and the nominal electric field strength (E=V/d, where V is the actual voltage across electrodes and d is the gap between electrodes) was about 2.5 kV/m. The electrochemical reactions in the solution are mainly due to the electrolysis of water for the following reasons: a) applied electrode voltage potential was greater than the electrolysis threshold (1.23 V) for water, and, b) salts were removed from the solution by extensive dialysis.

Anode: 2H₂O-4-e→4H⁺+O₂; Cathode: 4H₂O+4e→4OH⁻+2H₂.

This pH gradient produces three effects on the collagen solution. The first effect involves the charging of collagen molecules. As an ampholytic molecule, the net charge of collagen molecules near the cathode becomes negative and the net charge of molecules near the anode becomes positive. Therefore, molecules are repelled away from the cathode and the anode towards a plane between the two electrodes. Under the effect of the external electric field, collagen molecules migrate until reaching their pI (the isoelectric point, pI-9 for the collagen used here), where they have no net charge. This results in the entrapment and assembly of collagen molecules at the pI, as viewed by the compensated polarized optical imaging (FIG. 9, Panel B). The uniform and strong blue interference color indicates that aggregated and assembled collagen molecules are oriented strictly parallel to the slow axis of the gypsum waveplate (i.e., oriented parallel to the long axis of the collagen band). The second effect of pH gradient is the charge imbalance it imposes along the length of rod-like collagen molecules. Charged functional groups are almost uniformly distributed along the length of the collagen molecule, where the charge is pH dependent. Collagen molecules that are not perpendicular to the pH-gradient (i.e., obliquely oriented to electrodes' longer axes) will have the end terminal closer to the anode (or cathode) more positively (or negatively) charged relative to the other end of the terminal. Consequently, each electrode repulses the proximal end of the molecule more strongly than the distal end due to electrostatic force, driving the collagen molecules skewed to the electrodes to rotate. Assuming a linear variation of pH between the two electrodes (from 3 to 10 over 1 mm distance), the hydrogen ion concentration changes about 0.45% across a 300 nm distance (rod length of one collagen molecule). This change in pH along the length of one molecule seems to be a sufficient to rotate molecules parallel to electrodes. This rotary alignment of collagen molecules was evident by the birefringence of collagen solution in the region defined between the pI and cathode (FIG. 9, Panel C and supplementary video). The third effect of the pH gradient is the viscosity change in the solution. The solution on the anode side became less viscous within seconds of the application of the current. The acidic pH in the anode is known to dissolve collagen molecules, explaining the sudden reduction in viscosity. As a consequence, the anodic environment becomes more conducive to molecular migration and the movement of collagen towards the isoelectric point takes place within seconds without detectable birefringence. On the cathode side, the basic pH is conducive to molecular aggregation and fibrillogenesis, thereby increasing the viscosity of collagen solution. Accordingly, the movement of collagen towards its pI is slow on the cathode side and the rotary alignment of collagen molecules becomes visible. In summary, both the rotary alignment of molecules parallel to electrodes and the isoelectric focusing are the outcomes of pH gradient, electrostatic interaction between molecules and electrodes, and the ampholytic nature of rod-like collagen molecules. This controlled collagen self-assembly process is depicted schematically in FIG. 7, Panel C.

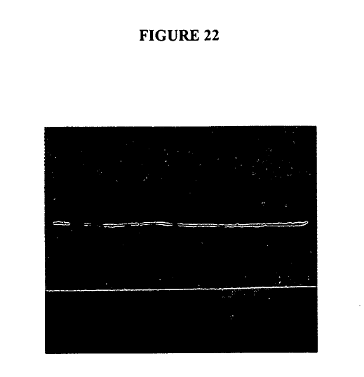

Once the congregation and self-assembly of aligned collagen molecules are complete in the vicinity of the isoelectric point (FIG. 9, Panel D insert), the resulting collagen band assumed a densely packed crystal-like conformation as indicated by the strong birefringence (FIG. 9, Panel D). The formation of the band took about one hour at the applied current density of 5.5 A/m² and electric field strength of 2.5 kV/m. Band formation times as short as 3 to 5 minutes were observed at higher current densities and nominal electric filed strength (e.g., voltage supply of 18 Volts with 1 MΩ. resistor in a similar set up). Bundles that are several inches long with a 50-400 μm diameter were fabricated by adjusting the electrode length, gap, current density, and nominal electric field (FIG. 22). These aligned collagen bundles were further crosslinked with a biocompatible natural crosslinking agent, genipin. An aligned, crosslinked collagen bundle was split open along its longer axis to expose the inside of the bundle. The SEM image showed that collagen bundle were oriented parallel to the longer axis of the bundle uniformly across the entire thickness (FIG. 9, Panel E).

To demonstrate the versatility of this electrochemical alignment technique for the generation of macroscale constructs of various shapes, a wire (0.01 inch diameter) was bent into a circular cathode and a point-source anode was placed at the center of the ring. This configuration yielded a ring-shaped collagenous construct close to the cathode region. Using the gypsum plate and crossed-polarizers, it was confirmed that the molecules were circumferentially oriented within the ring, as evident by the blue and the yellow interference colors in directions parallel and perpendicular to the slow axis of the gypsum plate, respectively (FIG. 9, Panel F).

### Example 2

### Electrochemically-Induced Controlled Collagen Self-Assembly Process

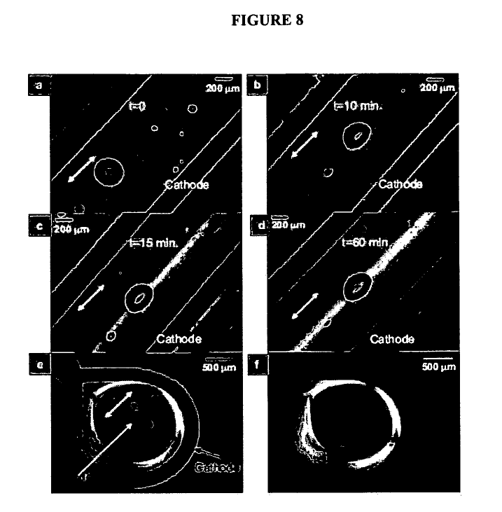

The controlled collagen self-assembly process depicted in FIG. 7, Panel C, is composed of rotary alignment, isoelectric focusing and pH induced self-assembly. The mechanism of this process is also evident by time-elapsed polarized optical images acquired during electrochemically induced fibrillogenesis (FIG. 8, Panels A-D). The migration of collagen molecules towards the isoelectric point compressively deformed the shape of intentionally induced air bubbles from circular to elliptical. Also, the width of the collagen band increased with time and became more birefringent as all molecules aligned and assembled at the isoelectric point. The crystalline quality and orientation of the resulting collagen band was confirmed by its uniform and strong blue interference color when the collagen construct was viewed under crossed-polarizers coupled with a gypsum plate positioned at 45° (FIG. 8, Panels C-D). This indicates that collagen molecules are strictly oriented parallel to the slow axis of the waveplate (i.e., oriented parallel to the long axis of the collagen band). At the applied electrical current level of 3.5 μA, it took about one hour for formation of the final band (FIG. 2, Panel D).

### Example 3

### Varying Geometry of Collagen Construct

To demonstrate the versatility of this electrochemical alignment technique for the generation of macroscale constructs of various shapes, a wire was bent into a circular cathode and a point-source anode was placed at the center of the ring (FIG. 14, Panel C). This configuration yielded a ring-shaped collagenous construct close to the cathode region. Using the gypsum plate and crossed-polarizers, it was confirmed that the molecules were circumferentially oriented within the ring, as evident by the blue and the yellow appearance in directions parallel and perpendicular to the slow axis of the gypsum plate, respectively (FIG. 8, Panel E). Without the gypsum plate, the collagen ring was highly birefringent under crossed-polarizers (FIG. 8, Panel F).

### Example 4

### Crosslinking of Collagen Samples

Randomly oriented collagen networks were made by gelling the salt-free collagen solution via addition of PBS at pH 7.4 and 37° C. treatment, without applying a voltage. The aligned collagen fibers, random collagen gel, and bleached tendon fibers were crosslinked in 15 ml of 0.625% genipin (Wako Pure Chemical, Osaka, Japan) in a 1×PBS solution at 37° C. for 3 days.

### Example 5

### Structure of Aligned Collagen Bundles Compared with Random Collagen and Tendon

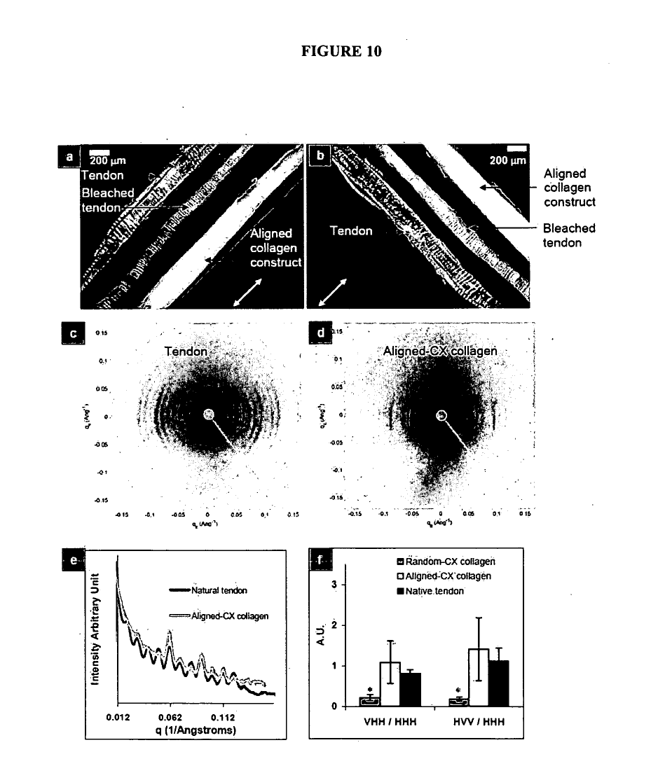

The structure of the aligned collagenous bundles formed by the parallel linear electrodes was compared with natural tendon fibers, since collagen fibrils of native tendons are mostly aligned uniformly. Native tendon bundles were obtained at the origin of the biceps muscle of a canine. The dissection was performed within several hours of euthanasia, which was conducted under the approval of the Purdue Animal Care and Use Committee. Native tendon fibers, as harvested, showed no uniform interference colors, due to the hindrance of collagen by other non-collageneous proteins and proteoglycans (FIG. 10, Panels A-B). The non-collagenous matter was partially removed by treating the tendon bundles with a 1% w/w NaOCl solution for 120 seconds. This treatment isolated the signal from the collagen fibers in the tendon, as evident by the emergence of interference colors with the presence of a microscale periodic banding, the so-called “crimp pattern” [Ottani et al., Micron, 32: 251-260 (2001), incorporated herein by reference]. The synthetic collagen bundle showed interference patterns similar to the bleached tendon, indicating that the degree of orientation for the natural and synthetic collagen bundles was comparable.

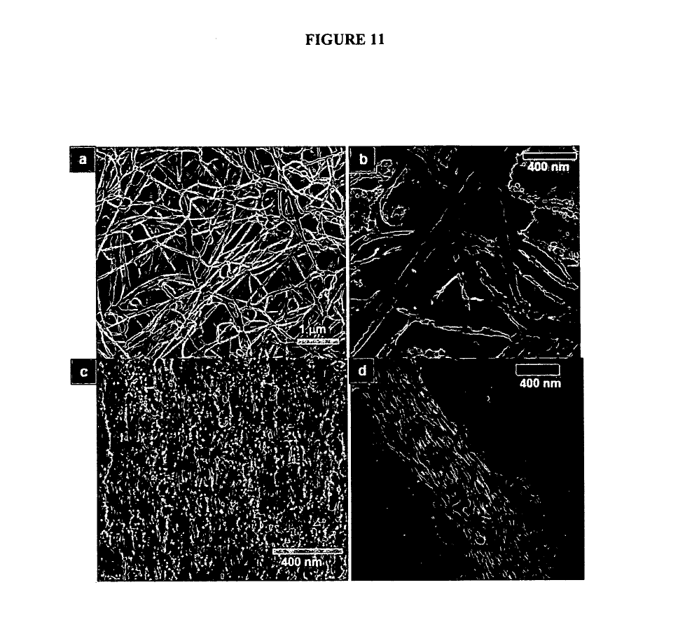

For comparison, the dialyzed collagen solution was cast as a gel without the application of an electric field to serve as random collagen control. The lack of orientation in gels formed without the electrochemical process was confirmed with scanning electron microscopy (SEM, FIG. 11, Panel A). On the other hand, electrochemically aligned collagen bundles were composed of uniformly oriented fibrils with diameters of 15-20 nm (FIG. 11, Panel C). After crosslinking the collagen with genipin, a natural biocompatible crosslinking agent, in a 1× phosphate buffered saline (PBS) solution, the orientation of the fiber bundles was retained.

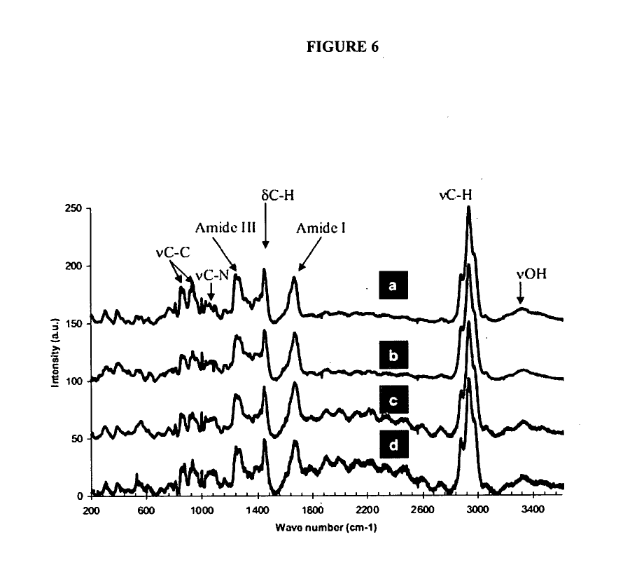

Although they were structurally different, the random collagen gel, aligned collagen construct, bleached tendon, and native tendon fibers were similar in their chemical composition. Raman spectra showed they are all composed of mainly collagen (FIG. 6).

### Small Angle X-Ray Scattering (SAXS):

SAXS patterns were collected using a three-pinhole SAXS camera (Molecular Metrology) with a microfocus x-ray source, an Osmic MaxFlux confocal X-ray optic, and a 2D Fujifilm image plate detector at a camera length of 1647 mm. The detector was calibrated using a silver behenate powder standard (q=0.107623 Å⁻). The main beam intensity was attenuated with a beam stop blocking all scattering below q=0.11 nm⁻¹). Intensity versus the magnitude of the scattering vector (q) plots were produced from a radial line plot of the 2D SAXS data along the direction of the fiber axis.

The intensity I(φ) of the strongest arc versus the azimuthal angle φ was obtained for tendon and the aligned crosslinked collagen. The integrals in Eq. (1) were carried out numerically and the order parameter S was obtained using Eq. (2). For an isotropic material, S=0, and for an ideal uniaxially oriented material, S=1.

$$\begin{matrix}〈 {cos}^{2} φ 〉 = \frac{{\int }_{0}^{π / 2} I ( φ ) sin ( φ ) {cos}^{2} φ d φ}{{\int }_{0}^{π / 2} I ( φ ) sin ( φ ) d φ} & ( 1 ) \\ S = 1 / 2 ( 3 〈 {cos}^{2} φ 〉 - 1 ) & ( 2 )\end{matrix}$$

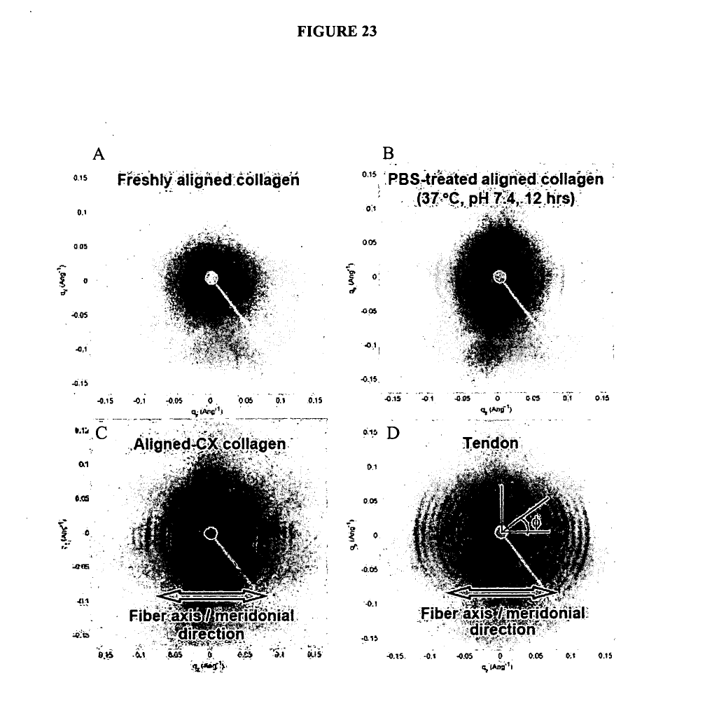

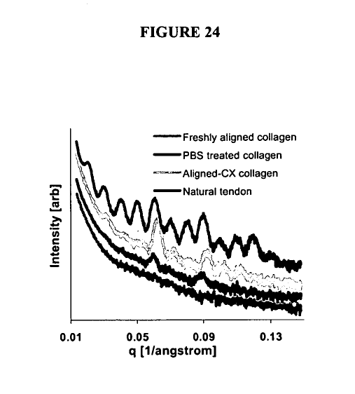

The 2D SAXS patterns (FIG. 23, Panels A-D; FIG. 24) showed the evolution of the D-banding as the electrochemically aligned collagen was subjected to subsequent PBS and genipin treatments. Freshly aligned collagen did not show the periodic arc pattern (FIG. 23, Panel A), indicating that there is a lack of D-banding despite that the molecules are oriented and aggregated at the isoelectric point. However, periodic arc pattern began to appear after the aligned collagen bundle was treated in PBS (FIG. 23, panel B). The periodic arc pattern became more pronounced after PBS-treated aligned collagen was crosslinked in genipin (FIG. 23, Panel C).

The presence of D-banding in the collagen material was assessed by small angle X-ray scattering (SAXS). The 2D SAXS patterns (FIG. 10, Panels C-D) showed the presence of a considerable long-range order along the fiber axis in both the native tendon and the aligned, crosslinked collagen samples. Upon comparison, it can be observed that the azimuthal angular breadth of the Bragg peaks in the aligned, crosslinked collagen sample was even smaller than that observed in the natural canine tendon (FIG. 10, Panel D). The sharp Bragg diffraction peaks observed along the fiber axis in the aligned, crosslinked collagen sample confirm a staggered axial packing of the collagen molecules. By comparison with the natural canine tendon, many peaks were observed at nearly the same q-value, shifted only slightly to smaller spacings (FIG. 10, Panel E; FIG. 24). The positions of the diffraction peaks observed from the natural tendon sample confirm a single periodic distance of 62.4 nm (Table 1). Thus, each observed peak occurs at a spacing of D=nπ2π/qₚₑₐₖ, where n is the order of the peak 1, 2, 3 etc. For natural tendon, peaks with n=2-13 were observed while the n=1 peak is behind the beam stop and was at too low of q to be observed by the apparatus. The positions of the peaks for aligned, crosslinked collagen sample can be explained by a slightly smaller D of 61.2 nm which is in the lower range of characteristic period banding (60-70 nm) found in type I collagen material. Peaks corresponding to n=3-13 were observed for aligned, crosslinked collagen bundles. Both for tendon and the electrochemically synthesized collagen the n=6 and n=9 were the most intense peaks, further indicating axial staggering of collagen molecules of the natural tendon and of the aligned, crosslinked sample are similar. The calculations from the 2D SAXS scattering data indicated that the aligned crosslinked collagen had an order parameter of S=0.84, whereas the natural tendon had S=0.69 (S=1 indicates perfect order, S=0 indicates random isotropic structure).

The lack of orientation in random collagen gels formed without the electrochemical process was confirmed with scanning electron microscopy (SEM) (FIG. 11, Panel A). Randomly oriented gels displayed the typical D-banding period around 63 nm as per TEM imaging (FIG. 11, Panel B). On the other hand, electrochemically aligned collagen bundles were composed of densely packed and uniformly oriented small fibers as evident by SEM imaging after serial dehydration (FIG. 11, Panel C). Un-crosslinked aligned collagen bundles were exposed by loosening the fiber network through maceration and tear while hydrated in water. TEM images showed ensembles of dense fibers (FIG. 11, Panel D). These fibers are 25-40 nm in diameter, a size similar to that reported for fibers of the cornea but much smaller than that of tendon. Fibers were composed of sub-fibrils of about 8 nm in diameter. The D-banding period of oriented collagen was not readily observable in TEM but its presence was confirmed by SAXS.

TABLE 1
Structural parameters obtained from the meridional reflections
in the measured SAXS diffraction pattern. From D = n − 2π/qₚₑₐₖ,
it can be deduced that the d period banding of both
collagen material are about 61-62 nm.

| | |
|---|---|
| Native tendon | Aligned collagen |

| Order n | q (1/nm) | d (nm) | q (1/nm) | d (nm) |
|---|---|---|---|---|
| 1 | — | 62.4 | — | 61.2 |
| 2 | 0.2025 | 31.2 | — | — |
| 3 | 0.3022 | 20.8 | 0.3161 | 19.9 |
| 4 | 0.4026 | 15.6 | 0.4004 | 15.7 |
| 5 | 0.5029 | 12.5 | 0.5222 | 12.0 |
| 6 | 0.6051 | 10.4 | 0.6205 | 10.1 |
| 7 | 0.7036 | 8.91 | 0.7212 | 8.7 |
| 8 | 0.8039 | 7.80 | 0.8289 | 7.6 |
| 9 | 0.9012 | 6.93 | 0.9294 | 6.8 |
| 10 | 1.0033 | 6.24 | 1.0233 | 6.1 |
| 11 | 1.1031 | 5.67 | 1.1286 | 5.6 |
| 12 | 1.1991 | 5.20 | | |
| 13 | 1.3099 | 4.80 | | |

TABLE 2
Tensile properties of collagen fiber bundles of different origin.
Reported values are the minimum and the maximum observation for a
given parameter. All samples are crosslinked at the same condition.
N = 10 per/group

| | Random-CX | Bleached | Aligned-CX | Native |
|---|---|---|---|---|
| | collagen gel | tendon-CX | collagen | tendon |
| Ultimate tensile | 0.8-2.5 | 6-30 | 24-88 | 57-148 |
| stress (MPa) | | | | |
| Ultimate tensile | 6-17% | 11-25% | 7-24% | 10-20% |
| strain | | | | |
| Tensile modulus | 9-38 | 40-174 | 277-671 | 448-977 |
| (MPa) | | | | |

### Example 6

### Tensile Tests

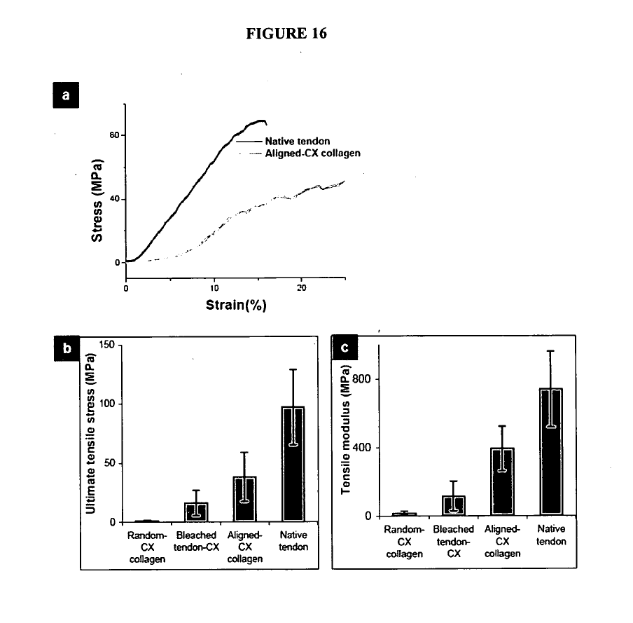

Tensile tests were carried out for four different groups of samples (N=10/group): aligned-CX collagen bundle, random, crosslinked collagen gel, bleached, crosslinked tendon bundles, and native tendon bundles. The crosslinking agent and conditions were identical for each sample. Typical stress strain curve of native tendon and aligned, crosslinked collagen shows a toe, heel and linear region (FIG. 16, Panel A). Aligned, crosslinked collagen bundles had 30-fold greater ultimate tensile strength and 25-fold greater tensile modulus than the random, crosslinked collagen gel (Table 2, FIG. 16, Panels B-C, p<0.05). The packing density and the orientational order of fibrils clearly contributed substantially to the mechanical strength of the bundle. The aligned, crosslinked collagen bundles also had two-fold greater ultimate tensile strength and tensile modulus than the bleached, crosslinked tendon (FIG. 16, Panels B-C, P<0.05). The native tendon bundles were the strongest and stiffest of the five groups. This indicates the degree of crosslinking and the presence of non-collagenous proteins and proteoglycans also contribute greatly to the strength and modulus of the tendon, in addition to the orientation factor. Nevertheless, aligned, crosslinked collagen bundles of the current study were the closest to the natural tendon among the remaining groups.

### Example 7

### High Resolution Scanning Electron Microscopy (SEM)

Random collagen gels (noncrosslinked) and aligned collagen bundles (non-crosslinked and crosslinked) were prepared using a procedure similar to that developed by Raub et al. for SEM imaging of collagen gels [Biophysical Journal, 92: 2212-2222 (2007), incorporated herein by reference]. Dried samples were mounted on holders and coated with Pt for 40 seconds prior to imaging (FEI NOVA nanoSEM, FEI Company, Oregon) using through-the-lens and Everhart-Thornley detectors at a 5 kV accelerating voltage.

The fibril area fraction for crosslinked and uncrosslinked aligned collagen constructs was determined using SEM image analysis. The uncrosslinked aligned collagen had a fibril area fraction of about 94%+/−3%. The crosslinked aligned collagen was greater than 94%. The random collagen compositions made by conventional methods had a fibril area fraction of 62.5%+/−11%.

### Example 8

### Transmission Electron Microscopy

Collagen samples were macerated in a depression slide using a scalpel and forceps in ultrapure water. About 10 μl of the supernatant was put on a transmission electron microscopy (TEM) grid and allowed to settle for 1 min Samples were stained with 1% Phosphotungstic acid and dried prior to imaging.

### Example 9

### SHG-NOE Analysis

Second harmonic generation—nonlinear optical ellipsometry (SHG-NOE) was performed using the discrete retardance nonlinear optical ellipsometer described previously [Review of Scientific Instruments, 78: 013106 (2007), incorporated herein by reference]. Three groups of samples were assessed: random, crosslinked collagen gel, aligned, crosslinked collagen construct, and native tendon fibers. Samples were air dried, placed between two glass slides, and measured in the transmission configuration (0° tilt). The SHG intensities were measured using different combinations of vertically (V) and horizontally (H) linearly polarized light for the incident and detected beams, with the vertical axis defined along the longer axis of bundles. The measured intensities were normalized to HHH. The intensity ratios were measured nine times for each sample (64 laser pulses per measurement). Reported standard deviations are from 27 measurements obtained for three separate samples and standard deviations are dominated by the sample-to-sample variance.

The SHG-NOE analysis of the native tendon and the random, crosslinked collagen samples served as reference points for assessing the order in the aligned, crosslinked collagen construct. The random, crosslinked collagen gel yielded values for the VHH/HVV and VVV/HHH ratios close to one (FIG. 10, Panel F). The first index indicates the SHG polarization component detected and the last two indices indicate the polarization state of the two incident photons, with V defined along the long fiber axis, if applicable. The ratios obtained for the aligned, crosslinked collagen construct suggested that these samples shared molecular-level structural similarities with the collagen in native tendon, and that they were significantly different (P<0.0001, N=27) from the random, crosslinked collagen gel. SHG9 NOE observations further indicated that the application of weak currents aligned the collagen molecules in a co-parallel orientation similar to that of the native tendon sample (i.e., both aligned and oriented, with the N-terminals of each collagen molecule pointing along the same direction as its neighbor in the synthetic aligned collagen bundle). Collagen molecules that are not perpendicular to the pH-gradient (i.e., parallel to electrodes) have the end terminal closer to the anode (or cathode) more positively (or negatively) charged relative to the other end terminal.

Consequently, each electrode repulses the proximal end of the molecule more strongly than the distal end due to electrostatic force, driving the collagen molecules skewed to the electrodes to rotate. Assuming a linear variation in pH between the two electrodes separated by 1 mm, the hydrogen ion concentration changes by ˜0.4% across a ˜300 nm fibril, which may be enough of a driving force to induce an alignment co-parallel to the electrodes when coupled to other intermolecular interactions, such as aggregation and liquid crystal formation.

### Example 10

### Mechanical Properties

The synthetic collagen bundles were tested in tension (N=10/group). Prior to tensile tests, samples were washed with deionized water, serially dehydrated in EtOH/H₂O mixtures, and air dried. Both ends of the dried bundles were fixed on plastic tabs using epoxy and rehydrated with a 1×PBS. Thicknesses of rehydrated samples were measured using a confocal microscope (Olympus FV1000) and the width was measured using a calibrated video-microscope. Samples were mounted on fixtures of an electromagnetically controlled materials testing machine (800 L, Testresources, Shakopee, Minn.) and loaded in tension to failure monotonically in displacement control (10 mm/min) Load was divided by the area of sample to obtain stress and the extension was normalized with the original gage length to calculate the strain. Elastic modulus was obtained by a linear regression fit. The regression line extended between the end of the toe region and the midpoint of the linear elastic region.

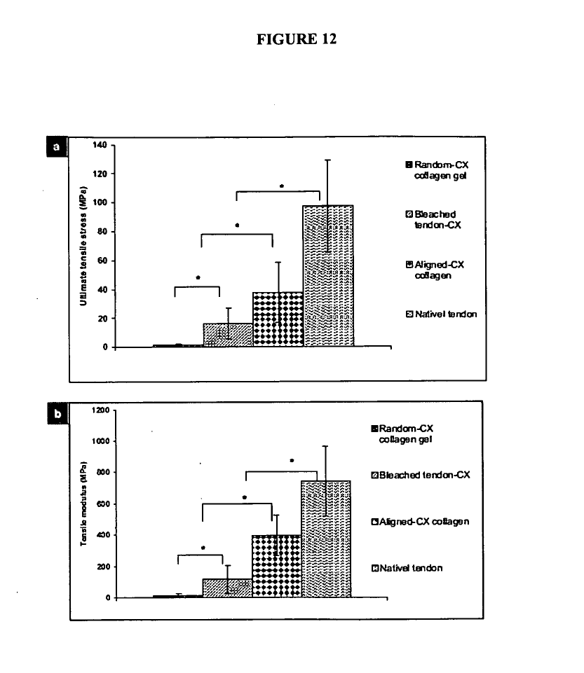

Tensile tests were carried out for four different groups of samples (N=10/group): aligned, crosslinked (CX) collagen bundle, random-CX collagen gel, bleached-CX tendon bundles, and native tendon bundles. The crosslinking agent and conditions were identical for each sample. Aligned-CX collagen bundles had 30-fold greater ultimate tensile strength and 25-fold greater tensile modulus than the random-CX collagen gel (Table 2, FIG. 12, Panel A, p<0.05). The packing density and the orientational order of fibrils contributed substantially to the mechanical strength of the bundle. The aligned-CX collagen bundles also had two-fold higher ultimate tensile strength and tensile modulus than the bleached-CX tendon (FIG. 12, Panels A-B, P<0.05). The native tendon bundles were the strongest and stiffest of the five groups. This indicates the degree of crosslinking and the presence of noncollagenous proteins and proteoglycans also contribute greatly to the strength and modulus of the tendon, in addition to the orientation factor. Nevertheless, aligned-CX collagen bundles of the current study were the closest to the natural tendon among the remaining groups. Ultimate tensile strain % for uncrosslinked compositions was about 1.2 to about 6% (data not shown).

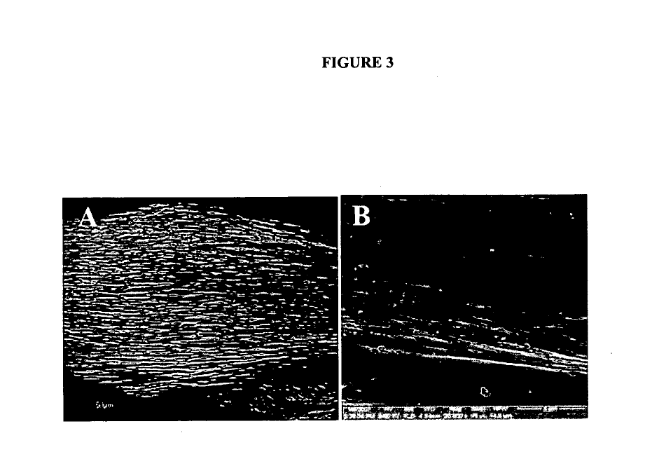

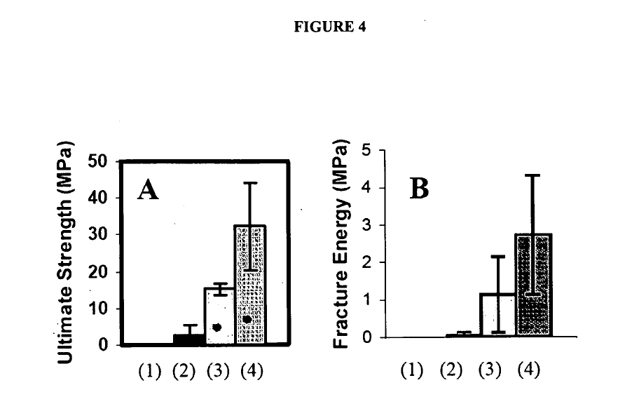

Formation of suprafibrillar collagenous bundles was evident in aligned gels as per confocal images (FIG. 3, Panel A) and SEM observations (FIG. 3, Panel B). The isotropic-uncrosslinked collagen gel was not solid enough to be tested mechanically (FIG. 4, panel A). On the other hand, anisotropic-uncrosslinked specimens were solidified enough to be tested mechanically. It was observed that the ultimate strength of anisotropic-crosslinked gels was twice higher than that of isotropic-crosslinked counterpart (FIG. 4, Panel A, 0.05<P<0.1). The difference in the energy to fracture was not significant.

Collagen molecules can be aligned into macroscopic scale across various hierarchical levels by applying weak electric fields to the solution. Anisotropic gels are strong enough to be tested even without crosslinking, underlying the importance of orientation. After crosslinking, the aligned collagen gels have much higher ultimate strength than non-aligned collagen gels.

Density measurements were determined for the samples using the “Sink-float” technique in salt solutions. The following measurements were obtained: uncrosslinked aligned collagen had a density of about 1.2 g/mL; crosslinked aligned collagen had a density greater than 1.2 g/mL (based on weight/volume measurement the density was about 1.4 g/mL+/−0.3); natural tendon had a density of about 1.12 g/mL; random collagen compositions made by conventional methods had a density of about 0.99 g/mL; and random collagen (crosslinked) had a density of about 1.04 g/mL.

Starting from a dilute collagen monomer solution, this electrochemical process leads to formation of a solid and oriented crystalline collagen construct. The formed high packing density and aligned collagen construct is table in water for up to two weeks or more. The collagen construct is still acid (e.g., 0.1N HCl) soluble and the collagen can be reconstituted. This process can also be used to concentrate collagen solutions or the process may be used to separate collagen form other proteins and biopolymers. The collagen construct can be stored and sterilized by immersing in 70% EtOH/H₂O solution for other applications.

### Example 11

### Cell Seeding and Attachment

### Progenitor Cells:

Rat bone marrow was extracted from the femurs of 40 days old Long-Evans rats immediately after they were sacrificed under IUCAC approval. The cells were extracted and cultured till confluence [Cell and Tissue Research, 248: 449-454 (1987), incorporated herein by reference]. Aligned collagen bundles were sterilized by immersing in 70% ethanol, washed with PBS, placed in the bottom of a 12-well plate and seeded with cells at a density of about 10⁵ cells/mL. After incubation for 24 hrs, recovered bundles were washed with PBS and treated with 3.7% formaldehyde and 0.1% Triton X-100 (Sigma). The collagen bundles were incubated with AlexaFluor 488 phalloidin dye (50 μl in 1 mL PBS at 1:20 dilution) at 37° C. to label the F-actin in cells and imaged (Olympus 2-photon Confocal Microscope).

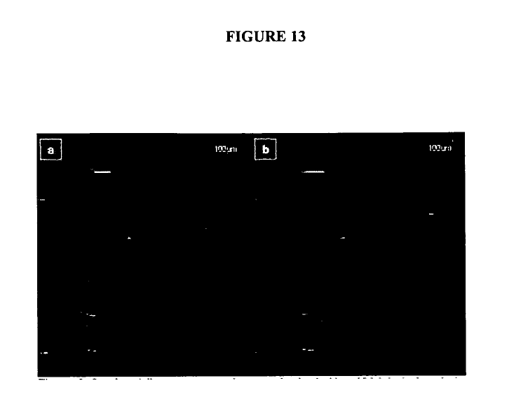

After 24 hours of seeding with bone marrow progenitors, the cells were readily attached to the surface of aligned collagen construct (FIG. 13). The attached cells appear to be stretched along the aligned collagen fiber direction, as evident by the fluorescence image of AlexaFluor 488 phalloidin labeled actin filament in cells. The spindle shaped morphology observed in native ligament or tendon can be duplicated in this novel oriented collagen construct. Such morphological compatibility will converge the genotypic and functional behavior of seeded cells towards those of native cells of fibrous ECMs with preferred orientations.

### Fibroblasts:

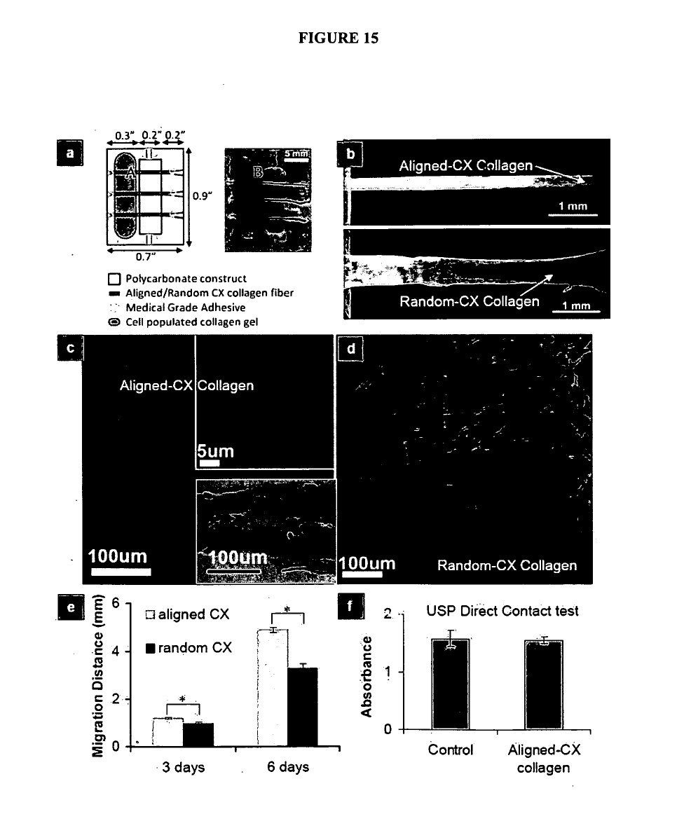

Rat tendon-derived fibroblasts were also grown on the aligned collagen constructs. At about passage 3 to 5 the crosslinked aligned collagen constructs had better fibroblast cell migration and proliferation compared to crosslinked random collagen constructs (n=3, migration rate ˜1 mm/day, FIG. 15, Panel E). Based on the direct contact test, the aligned collagen did not show cytotoxicity against tendon-derived fibroblasts (FIG. 15, Panel F).

### Example 12

### Cell Migration Assay and Direct Contact Test

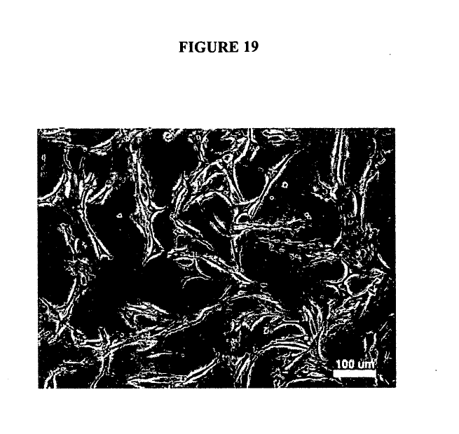

Achilles tendons of a 50 days old male Long-Evans rat (sacrificed under the approval of Purdue Animal Care and Use Committee) were harvested aseptically. The tendon specific fibroblasts were extracted and cultured by following established methods. The cells demonstrated typical spindle-shaped fibroblast morphology (FIG. 19) and the cells were passed between 3-5 times before being used.

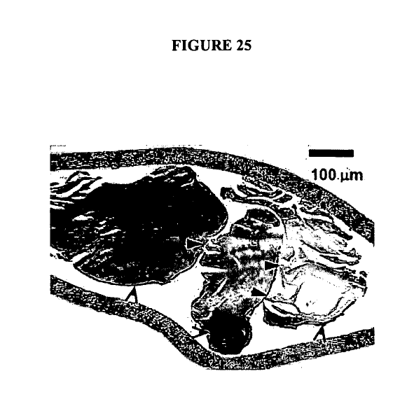

To demonstrate the potential of aligned-CX collagen for tendon/ligament tissue engineering, rat tendon-derived fibroblasts were cultured. A cell migration assay was conducted on single aligned-CX collagen bundle (N=3) and strips of random-CX collagen (N=3). Fibroblasts were cast inside a collagen gel, loaded on one end of the bundle (FIG. 15, Panel A) and cultured. An experiment was also performed wherein a collagen gel was loaded on each end of the collagen bundle or fiber. The migration of cells was visualized by fluorescence microscopy (FIG. 15, Panel B). It was observed that tendon fibroblasts migrated faster on aligned-CX collagen bundle than on random-CX collagen fiber (FIG. 15, Panels B and E). Cells in aligned-CX collagen bundle were stretched and elongated along the axis of orientation of collagen molecules (i.e. longer axis of collagen bundles) as per fluorescent images of the actin filaments of the cytoskeleton (FIG. 15, Panels C-D). Also, the cells were more ordered and closely populated on aligned bundles. H&E stained histological slides showed that the nuclei of fibroblasts on aligned-CX collagen bundles were also elliptical, stretched and oriented along the bundles' longer axes, a morphology similar to those reported for tenocytes in native tendon fibers (FIG. 15, Panel C, inset; nuclear aspect ratio=major length/minor length=2.3±0.3). The migration rate of fibroblast cells on 3D bundles was determined to be about 0.5 mm/day. Histological images of braided bundle groups revealed that cells were able to migrate and infiltrate the space between bundles (FIG. 25).

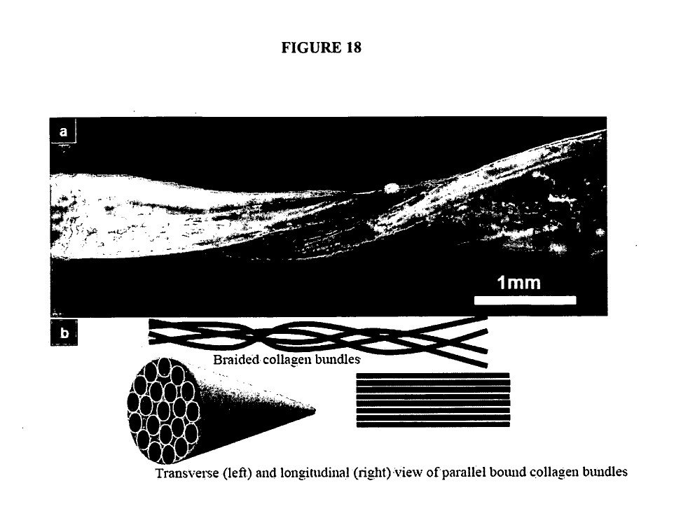

The bundles produced in the current study measured several hundred microns in diameter and several inches in length. The bundles themselves are densely packed and would not allow the migration of cells within; however, the envisioned practical use of these bundles for ligament or tendon replacement would involve grouping several bundles together. Seeded cells would then populate the space between the bundles. A 3D bundle network was constructed by twisting three aligned-CX bundles together to form a 3D construct (approximately 0.5 mm diameter, 7 mm length). These dimensions correspond to the dimensions of a secondary fascicle in tendon's hierarchical organization. These bundle constructs were then subject to the migration assay described earlier. Confocal fluorescent images of bundle groups revealed that cells were able to migrate and infiltrate the space between bundles (FIG. 18).

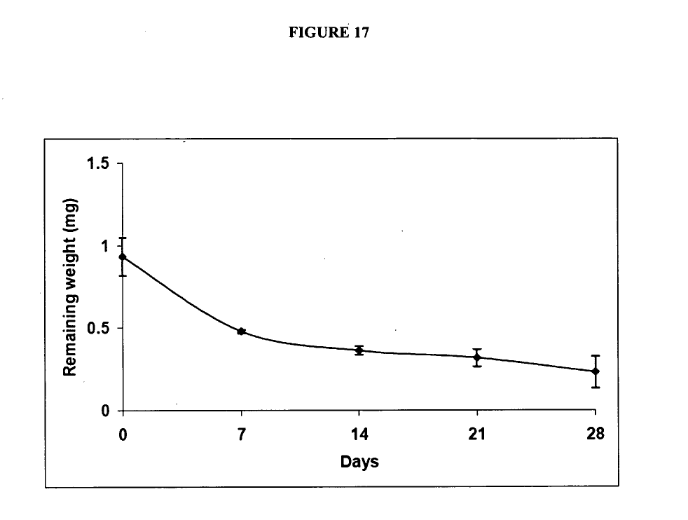

Universal standard procedure (USP) direct contact test was used to investigate the toxicity of the aligned-CX collagen to the tendon-derived fibroblasts. The number of viable cells which were in direct contact with aligned-CX collagen bundle was not significantly different from controls (FIG. 15, Panel F), confirming the reported biocompatibility of various genipin crosslinked collagen tissues. An in vitro degradation study indicated that aligned collagen in uncrosslinked form was completely degraded after 24 hrs in the presence of bacterial collagenase type I. Crosslinking with genipin greatly reduced the degradability of collagen construct and the aligned-CX collagen maintained about 28% of its initial weight after 28 days (FIG. 17).

### Example 13

### In Vitro Degradation Study

One mg of aligned-CX collagen was immersed in 200 μL of digest solution and incubated in an orbital shaker at 37° C. at 100 rpm. The digest solution (pH 7.4) contained 0.1 M tris-base, 0.25M CaCl2 and 125 U/mL of Type I collagenase (Sigma) from Clostridium histolyticum. At the designated time points, the remaining collagen bundle was taken out, washed, serially dehydrated and weighed using an ultra-sensitive balance. The dried bundle was immersed in freshly prepared digest solution again for the other time points (see FIG. 18).

### Example 14

### Cell Culture, Cell Migration, Direct Contact Test, and Cell Bundle Study

### Cell Culture

Achilles tendons of a 50 days old male Long-Evans rat (sacrificed under the approval of Purdue Animal Care and Use Committee) were harvested aseptically. The tendons were sliced into two pieces, disbanded with a sterile scalpel and each piece was incubated in a 6-well plate with the growth medium. The growth medium was composed of α-MEM (Sigma, M4526) supplemented with 10% FBS (Sigma, F6178), 2 mM L-Glutamine (Sigma, G7513), 100 U/ml Penicillin and 100 μg/ml Streptomycin (Invitrogen, 15140-122), 1.5 μg/ml Fungizone (Gibco, 15290-018). The tendon specific fibroblasts leaving the tendon fragments and attaching the tissue culture treated plastic reached 80-90% confluence on the 7th day. The tendon fragments were removed and the cells were passed to T-75 flasks and incubated till 80-90% confluence after each passage. The cells (FIG. 19) were passed 3 to 5 times before being used in the direct contact test and the cell migration assay.

### Cell Migration Studies

The cell migration assay used in this study was modified from Cornwell et al. [J. of Biomedical Materials Research Part A, 80A: 362-371 (2006)], where the migration of human derived fibroblasts on self-assembled collagen threads extruded from solutions of type I collagen molecules cross-linked with different techniques was investigated. The cell migration assay constructs were made out of 2 mm thick polycarbonate sheet. The constructs had a central gap on which the genipin cross-linked aligned and random collagen bundles were suspended. The collagen fibers were adhered to the constructs at the ends with medical grade adhesive (Loctite 4851), which has relatively high viscosity to minimize the wicking of the adhesive onto the collagen fibers. In order to prevent the cells from populating on the polycarbonate construct and reaching the far ends of the collagen fibers, vertical groves were machined on the upper and lower connecting polycarbonate sections. These groves were filled with a lower viscosity medical grade adhesive (Loctite 4014), which hinders the attachment of the cells drastically. The constructs were sterilized in 70% ethanol for 24 hours and air dried in the laminar flow hood before seeding. The cells were seeded onto the construct in a type-I collagen gel lattice. The collagen gel was prepared by mixing 8 parts type-I collagen (6 mg/ml) (Nutragen, USA) with 1 part 10×MEM (Sigma, M0275) and balancing the pH at 7.4 with 0.1N NaOH. The rat-tendon derived fibroblasts cultured up to 80-90% confluence were tyrpsinized, centrifuged at 1200 RPM for 5 minutes and the pellet was dispersed in the collagen gel at a concentration of 30×105 cells/ml gel. The gel containing the cells was then poured on the air dried constructs and incubated in CO₂-free incubator for 45 minutes. Growth media was added to the Petri dishes, which contained the migration assay constructs, sufficient to submerge the collagen fibers and a portion of the collagen gel lattice and incubated for up to 6 days. The constructs were stained with Alexa Fluor 488 Phalloidin (Molecular Probes) on the 3rd day and the 6th day and imaged under the fluorescent microscope.

### Direct Contact Test

The rat-tendon derived fibroblasts were incubated in a 12-well plate for 2 days before the aligned, genipin crosslinked collagen fibers were placed in the wells containing the cells. The aligned, genipin crosslinked collagen fibers were cut into 10 mm length, sterilized in 70% ethanol for 24 hours, rinsed 3 times with 1×PBS and once with the growth medium before they were placed in 4 wells (4 fibers per well). The cells in direct contact with the aligned crosslinked collagen fibers and the controls (cells only) were incubated an additional 24 hours before the number of viable cells was measured with CellTiter 96 AQ (Promega) cell proliferation assay.

### Cell Bundle Study

Four aligned-CX fibers were twisted together and glued on polycarbonate construct similar to the previous migration study. A procedure similar to previous cell migration study was used except that cell sources were migrated from both ends of the fiber bundle. The construct was taken out after 7 days incubation and examined for cell morphology, migration and histological analysis.

### Example 15

### Mineralized Constructs

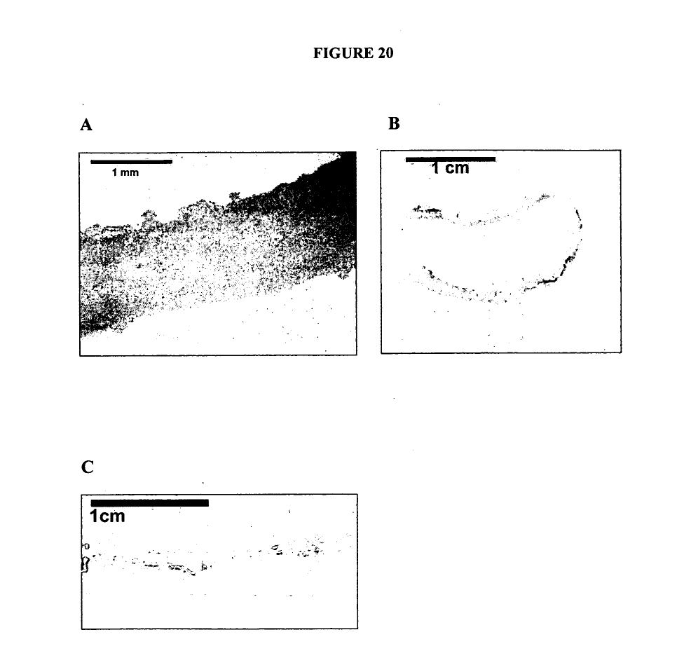

Calcium phosphate formation was achieved by mixing equal volumes of 9 mM CaCl₂ solution in Tris buffer (pH 7.4) and 4.2 mM K₂HPO₄ solution in Tris buffer (pH 7.4), to a final concentrations of 4.5 mM calcium and 2.1 mM phosphate. The aligned collagen construct was put in the above solution mixture and incubated to allow for mineralization for 6 days. The mineralized collagen construct was taken out and dried (FIG. 20). The hydroxyapatite mineralized aligned collagen fiber without crosslinking is shown in FIG. 20, Panels A and B (Note: Mineralized aligned collagen is not transparent). Aligned collagen bundles are shown in FIG. 20, Panel C (Note: Collagen bundles are transparent).

### Example 16

### Electrostatic Interactions and pH Gradient

An experiment was conducted with separation distances of 1 mm, 2 mm, 7 mm, and 15 mm, while the actual voltage applied across the electrodes was maintained the same (2.5 V). The alignment of collagen diminished as the separation increased. The time for the aligned band to appear at 7 mm was longer than at 1 mm or 2 mm. At 15 mm separation, the aligned collagen band was not observed within 24 h. At larger separation, the pH gradient was smaller and the “electric dipole” acting upon each collagen monomer became less pronounced. Also, the nominal electric field strength became smaller. These results show the use of electrostatic forces and a steep pH gradient for rotational alignment of collagen monomers.

### Example 17

### Statistical Analysis

Significance of differences between groups was tested with a non-parametric one-way ANOVA (Kruskal Wallis) and if the difference was significant at p<0.05, then differences between any two groups were further assessed by a non-parametric Mann-Whitney U-Test with the level of significance set at p<0.05 (Minitab, College Station, Pa.).

### Example 18

### Cell Growth on Anisotropic Collagen Constructs

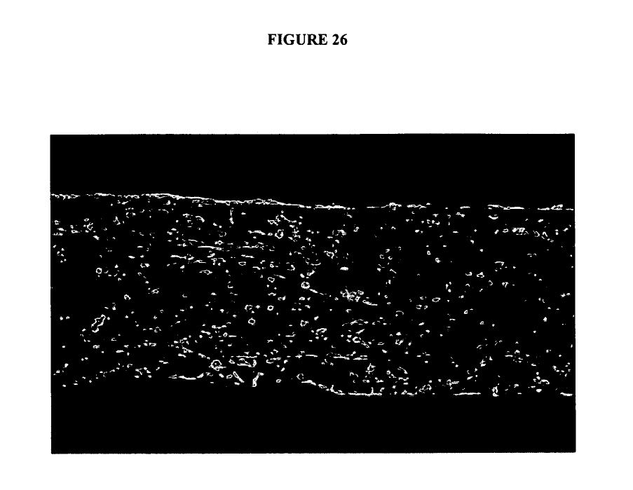

Schwann cells attach and migrate on anisotropic collagen constructs, prepared as herein described. Schwann cells were cultured on anisotropic collagen constructs using an in vitro cell growth assay (FIG. 26). The cells grown on the anisotropic collagen constructs attached to the collagen construct. The cells also stretched and oriented along the longer axis of anisotropic collagen construct. This data indicates that the construct will induce directionality to cell orientation and guide neural growth along the oriented collagen bundle axis.

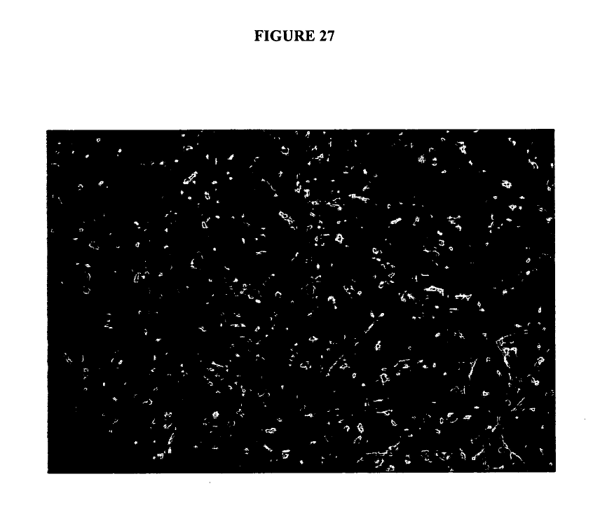

Schwann cells cultured on randomly oriented collagen attached to the collagen. However, unlike the cells grown on anisotropic collagen constructs described above, the cells grown on randomly oriented collagen neither stretched nor oriented along a specific direction (FIG. 27).

What is claimed is:

1. An engineered graft construct comprising aligned collagen fibrils wherein the construct is anisotropic, and wherein the fibril area fraction is about 80% to about 100%.

2. The graft construct of claim 1 wherein the collagen is crosslinked.

3. The graft construct of claim 1 wherein the collagen is uncrosslinked.

4. The graft construct of claim 1 wherein the fibril area fraction of the composition is about 83% to about 100%.

5. The graft construct of claim 1 wherein the fibril area fraction of the composition is about 86% to about 100%.

6. The graft construct of claim 1 wherein the fibril area fraction of the composition is about 90% to about 100%.

7. The graft construct of claim 1 wherein the density of collagen in said graft construct is at least 1.1 g/mL.

8. The graft construct of claim 1 wherein said construct is selected from the group consisting of a thread, a rope, a ring, a sheet, a tube, and combinations thereof.

9. The graft construct of claim 8 wherein said construct is woven or braided.

10.-13. (canceled)

14. An engineered graft construct comprising aligned collagen fibrils wherein the construct is anisotropic, and wherein the ultimate tensile stress of said graft construct is about 0.5 MPa to about 150 MPa.

15. The graft construct of claim 14 wherein the collagen is crosslinked.

16. The graft construct of claim 14 wherein the collagen is uncrosslinked.

17. The graft construct of claim 14 wherein the ultimate tensile stress of said graft construct is about 24 MPa to about 88 MPa.

18. The graft construct of claim 14 wherein the ultimate tensile stress of said graft construct is about 1 MPa to about 5 MPa.

19. The graft construct of claim 14 wherein the ultimate tensile stress of said graft construct is about 4 MPa to about 40 MPa.

20.-26. (canceled)

27. An engineered graft construct comprising aligned collagen fibrils wherein the construct is anisotropic, and wherein the ultimate tensile strain of said graft construct is about 0.5% to about 30%.

28. The graft construct of claim 27 wherein the collagen is crosslinked.

29. The graft construct of claim 27 wherein the collagen is uncrosslinked.

30. The graft construct of claim 27 wherein the ultimate tensile strain percent of said graft construct is about 0.5% to about 10%.

31. The graft construct of claim 27 wherein the ultimate tensile strain percent of said graft construct is about 1% to about 10%.

32.-91. (canceled)
